(19) United States
(12) Patent Application Publication
(10) Pub. No.: US 2019/0106721 A1
(43) Pub. Date: Apr. 11, 2019

(54) Method for the fermentative production of L-amino acids

(71) Applicant: Evonik Degussa GmbH, Essen (DE)

(72) Inventors: Thomas BEKEL, Halle (Westf.) (DE); Georg Thierbach, Bielefeld (DE); Kornelia Voß, Steinhagen (DE)

(73) Assignee: Evonik Degussa GmbH, Essen (DE)

(21) Appl. No.: 16/149,285

(22) Filed: Oct. 2, 2018

(30) Foreign Application Priority Data
Oct. 5, 2017 (EP) 17194981.1

(51) Int. Cl. C12P 13/08; C12P 13/06

(52) U.S. Cl. CPC C12P 13/08; C12P 13/06

(57) ABSTRACT

A method for the fermentative production of an L-amino acid, selected from L-lysine, L-threonine and L-isoleucine, includes cultivating a bacterium of the genus Corynebacterium having the ability to excrete said L-amino acid in a suitable medium under suitable conditions. The method also includes accumulating said L-amino acid in the medium to form an L-amino acid containing fermentation broth. Said bacterium comprises a polynucleotide coding for a polypeptide conferring a resistance to lincosamides which has been modified by deleting or replacing at least parts of said polynucleotide and at least one copy of a gene coding for a feedback resistant aspartokinase.

### CROSS-REFERENCE TO RELATED APPLICATIONS

This application claims the benefit of European Application No. EP 17194981, filed on Oct. 5, 2017, the entirety of which is incorporated herein by reference.

### REFERENCE TO A SEQUENCE LISTING

The instant application contains a Sequence Listing which has been filed electronically in ASCII format and is hereby incorporated by reference in its entirety. Said ASCII copy, created on Sep. 20, 2018 is named 201700286A_Seq.txt and is 96,309 bytes in size.

### BACKGROUND OF THE INVENTION

### Field of the invention

The present invention relates to a method for the fermentative production of an L-amino acid, selected from L-lysine, L-threonine and L-isoleucine, comprising the steps of cultivating a bacterium of the genus Corynebacterium having the ability to excrete said L-amino acid in a suitable medium under suitable conditions and accumulating said L-amino acid in the medium to form an L-amino acid containing fermentation broth.

### Description of the related art

L-Amino acids are used in human medicine, in the pharmaceutical industry, in the food industry and particularly in nutrition of animals.

L-amino acids such as, for example, L-lysine, are produced by fermentation of strains of the genus Corynebacterium, in particular Corynebacterium glutamicum. Because of the great economic importance, work is continually being done on improving the production methods. Improvements may relate to the fermentation technology such as e. g. stirring and supplying oxygen, or to the composition of the nutrient media e.g. the sugar concentration during fermentation, or to the processing of the fermentation broth to a suitable product form by e. g. drying and granulating the fermentation broth or ion exchange chromatography or may relate to the intrinsic performance properties of the microorganism itself.

The methods used for improving the performance properties of these microorganisms are those of mutagenesis, selection and screening of mutants. The strains obtained in this way are resistant to anti-metabolites or are auxotrophic for metabolites of regulatory importance, and produce L-amino acids. A well-known anti-metabolite is the L-lysine analogue S-(2-aminoethyl)-L-cysteine (see e.g. Tosaka et al.: Agricultural and Biological Chemistry 42(4), 745-752, (1978)). Methods of recombinant DNA technology have likewise been used for a number of years for strain improvement of L-amino acid-producing strains of the genus Corynebacterium, in particular Corynebacterium glutamicum, by modifying, i.e. enhancing or attenuating, individual L-amino acid biosynthesis genes and investigating the effect on L-amino acid production. The nucleotide sequences of the chromosomes of various bacteria or strains resp. of the genus Corynebacterium and of the species Corynebacterium glutamicum, and their analysis have been disclosed. This information is available at publicly accessible data bases and may be used for strain development purposes. One such data base is the GenBank data base of the NCBI (National Center for Biotechnology Information, U.S. National Library of Medicine 8600 Rockville Pike, Bethesda Md., 20894 USA).

During the annotation procedure for a sequenced chromosome of an organism identified structures such as e. g. genes or coding sequences are furnished with a unique identifier called locus_tag by the supplier of the information to the data base.

The nucleotide sequence of the Corynebacterium glutamicum ATCC13032 chromosome and its analysis was described by Ikeda and Nakagawa (Applied Microbiology and Biotechnology 62, 99-109(2003)) and in EP1108790 A2. It is available at the NCBI under accession number NC_003450. Locus_tag NCgl2592 identifies a sequence coding for a “major facilitator superfamily permease”. The nucleotide sequence of the Corynebacterium glutamicum R chromosome and its analysis was described by Yukawa et al. (Microbiology 153(4)1042-1058 (2007)). It is available at the NCBI under accession number AP009044. Locus_tag cgR_2586 identifies a sequence coding for a hypothetical protein.

Lv et al. (Journal of Bacteriology 194(3), 742-743 (2012) describe the sequencing of the chromosome of Corynebacterium glutamicum ATCC14067, a strain formerly referred to as Brevibacterium flavum. It is available at the NCBI under accession number AGQQ02000001 and AGQQ02000002. Locus_tag KIQ_001365 identifies a sequence coding for an “MFS transporter permease”.

The nucleotide sequence of the chromosome of Corynebacterium glutamicum ATCC13869, a strain formerly referred to as Brevibacterium lactofermentum, and its analysis were disclosed by Chen et al. at the NCBI under accession number NZ_CP016335. Locus_tag BBD29_13115 identifies a sequence coding for an “MFS transporter permease”.

Kim et al. (Molecules and Cells 12(1), 112-116 (2001)) identified the lmrB gene of Corynebacterium glutamicum, which encodes a protein conferring resistance to lincosamides, such as lincomycin and clindamycin. The strain of Corynebacterium glutamicum used by the authors is referred to as ASO19E12. Strain ASO19E12 originates from ATCC13059 (see; Follettie et al; Journal of Bacteriology 175(13), 4096-4103 (1993)). Kim et al suggested that the resistance to lincomycin is an energy dependent efflux system. The nucleotide sequence of said lmrB gene and the encoded amino acid sequence is available at the GenBank data base of the NCBI under accession number AF237667. It is also shown under SEQ ID NO:1 of the sequence listing.

Lincomycin is a lincosamide antibiotic produced by Streptomyces lincolnensis. The CAS (Chemical Abstract Service) registry number is 154-21-2. The CAS registry number of the corresponding HCl salt is 859-18-7. Clindamycin is a chlorinated derivative of lincomycin. The CAS registry number is 18323-44-9. The CAS registry number of the corresponding HCl salt is 21462-39-5. Summaries concerning antibiotics may be found inter alia in the text book of Jason C. Gallagher and Conan MacDougall (Antibiotics Simplified, 2^{nd} edition, Jones & Bartlett Learning, 2012). Corynebacterium, particularly Corynebacterium glutamicum, contains a gene in its chromosome encoding a polypeptide conferring resistance to lincosamide antibiotics selected from lincomycin and clindamycin.

Nakagawa discloses under GenBank accession number BAC00079 the encoded amino acid sequence of a protein defined as “Permeases of the major facilitator superfamily” of Corynebacterium glutamicum of ATCC13032. It is also shown under SEQ ID NO:2 of the sequence listing.

The amino acid sequences of entries AF237667 and BAC00079 were found to be identical over the full length. Said two amino acid sequences were also found to be identical with the amino acid sequence of the protein of ATCC13032 as disclosed in SEQ ID NO:2 of WO2001000804 A2 (see also GenBank accession number CAC26288). The amino acid sequence of SEQ ID NO:2 of WO2001000804 A2 is identical with the amino acid sequence of SEQ ID NO:8 of the sequence listing of the present document.

Nishio et al. disclose under GenBank accession number BAV24312 the encoded amino acid sequence of a protein defined as “lincomycin resistance protein lmrB” of Corynebacterium glutamicum ssp. lactofermentum strain AJ1511. It is also shown under SEQ ID NO:3 of the sequence listing in the present document.

Chen et al. disclose under GenBank accession number ANU34602 the encoded amino acid sequence of a protein defined as “MFS transporter permease” of Corynebacterium glutamicum ATCC 13869. This strain was formerly referred to as Brevibacterium lactofermentum. This sequence is also shown under SEQ ID NO:4 of the sequence listing in the present document.

The amino acid sequences of entries ANU34602 and BAV24312 were found to he identical over the full length. When compared to the amino acid sequence of the corresponding polypeptide of ATCC13032 the identity was found to be 99.2%.

Yukawa et al. disclose under GenBank accession number BAF55600 the encoded amino acid sequence of a “hypothetical protein cgR_2586” from Corynebacterium glutamicum R having a region named “MFS”. The amino acid sequence disclosed in BAF55600 is also shown under SEQ ID NO:5 of the sequence listing. Its identity to the corresponding amino acid sequence from ATCC13032 was found to be 96.7%. The term “MFS” is the abbreviation for “Major Facilitator Superfamily”. According to the conserved domain database at the NCBI (see database entry cd06174) the term denotes a large and diverse group of secondary transporters that includes uniporters, symporters, and antiporters, which facilitate the transport across cytoplasmic or internal membranes of a variety of substrates including ions, sugar phosphates, drugs, neurotransmitters, nucleosides, amino acids, and peptides.

Lv et al. disclose under GenBank accession number KEI24239 the encoded amino acid sequence of a protein from Corynebacterium glutamicum ATCC14067 (formerly referred to as Brevibacterium flavum) defined as “MFS transporter permease”. It is also shown under SEQ ID NO:6 of the sequence listing. Its identity to the corresponding amino acid sequence from ATCC13032 was found to be 97.9%.

A summary of the findings is shown in table 1.

TABLE 1
Comparison of the encoded amino acid sequences of LmrB polypeptides
of various strains of Corynebacterium glutamicum with the encoded amino
acid sequence of the LmrB polypeptide of ATCC13032 (GenBank
accession number BAC00079) by sequence alignment using Clustal
W (Larkin et al.: Clustal W and Clustal X version 2.0. In:
Bioinformatics 23, 2947-2948 (2007)).

| | Accession | | Identical Amino | |
|---|---|---|---|---|
| Strain | Number | Length* | Acids | % Identity |
| ASO19E12 | AF237667 | 481 | 481 | 100.0 |
| AJ1511 | BAV24312 | 481 | 477 | 99.2 |
| ATCC13869 | ANU34602 | 481 | 477 | 99.2 |
| R | BAF55600 | 481 | 465 | 96.7 |
| ATCC14067 | KEI24239 | 481 | 471 | 97.9 |

*number of amino acid residues

WO2001000804 A2 discloses various genes encoding stress resistance and tolerance proteins from Corynebacterium glutamicum ATCC 13032 and their use for the modulation of production of fine chemicals. The lmrB gene and the encoded polypeptide are disclosed under identifier RXA01524 and SEQ ID NO: 1 and 2 of said application. It is suggested to use drug or antibiotic resistance genes, e.g. the lmrB gene, as a tool for the discovery of new antibiotics. It is also suggested to express or overexpress said gene in a suitable host to create an organism, which may be used to screen for novel antibiotic compounds (see page 54 and 95 97). WO2001000804 A2 further discloses Corynebacterium glutamicum in which stress, resistance and tolerance (SRT) proteins are disrupted, e.g. lmrB (i.e. SEQ ID NO: 2 which is identical to SEQ ID NO: 8 in the present document). In a preferred embodiment the microorganism is also used for the production of amino acids, e.g. L-lysine.

### BRIEF SUMMARY OF THE INVENTION

An object of the present invention is to provide new measures for the fermentative production of L-amino acids selected from L-lysine, L-threonine and L-isoleucine by bacteria of the genus Corynebacterium, preferably of the species Corynebacterium glutamicum.

This and other objects have been achieved by the present invention, the first of which includes a method for the fermentative production of an L-amino acid, selected from L-lysine, L-threonine and L-isoleucine, comprising the steps of

a) cultivating a bacterium of the genus Corynebacterium having the ability to excrete said L-amino acid in a suitable medium under suitable conditions,

b) accumulating said L-amino acid in the medium to form an L-amino acid containing fermentation broth,

wherein in said bacterium a polynucleotide coding for a polypeptide, which is at least 90% identical to the amino acid sequence of SEQ ID NO:8 and which confers a resistance to lincosamides, selected from lincomycin and clindamycin, is modified by deleting or replacing at least parts of said polynucleotide and wherein said bacterium comprises at least one copy of a gene coding for a feedback resistant aspartokinase.

### BRIEF DESCRIPTION OF THE SEVERAL VIEWS OF THE DRAWINGS

FIG. 1 is a map of plasmid pVWEx1 (Example 7).

FIG. 2 is a map of plasmid pVWEx1_ImrBATCC14067 (Example 7).

### DETAILED DESCRIPTION OF THE INVENTION

Any ranges mentioned herein below include all values and subvalues between the lowest and highest limit of this range.

The bacterium of the genus Corynebacterium, preferably of the species Corynebacterium glutamicum, used in the method according to the present invention has the ability to excrete an L-amino acid selected from L-lysine, L-threonine and L-isoleucine, preferably L-lysine. In this bacterium a polypeptide conferring resistance to lincosamides, selected from lincomycin and clindamycin, encoded by an lmrB gene is modified by deleting or replacing at least parts of said polynucleotide resulting in an elimination or a switching off said polypeptide.

The polypeptide encoded by the modified polynucleotide and having the activity of conferring resistance to lincosamides, selected from lincomycin and clindamycin, preferably lincomycin, comprises an amino acid sequence, which is at least 90%, at least 95%, particularly preferred at least 96% very particularly preferred at least 97%, at least 98% or at least 99% or 100%, preferably 99.8% identical to the amino acid sequence of SEQ ID NO:8.

In the method according to the present invention it is furthermore necessary that the bacterium comprises at least one copy of a polynucleotide coding for a feedback resistant aspartokinase. A feedback resistant aspartokinase means an aspartokinase variant which is less sensitive or desensitized with respect to inhibition of its activity by L-lysine and/or L-threonine.

This feedback resistance of a particular aspartokinase variant can be determined by measuring its activity in the presence of mixtures of L-lysine and L-threonine, e.g. 10 mM each, or mixtures of the L-lysine analogue S-(2-aminoethyl)-L-cysteine and L-threonine, e.g. 50 mM S-(2-aminoethyl)-L-cysteine and 10 mM L-threonine, in comparison to the wild form of the enzyme, which is contained in wild strains like for example ATCC13032, ATCC14067 and ATCC13869.

The EC number for aspartokinase is EC 2.7.2.4. Descriptions of polynucleotides of Corynebacterium glutamicum encoding a feedback resistant aspartokinase polypeptide variant are for example given in U.S. Pat. Nos. 5,688,671, 6,844,176 and 6,893,848. A summarizing list can be found inter alia in WO2009141330 A1 (US2009311758 A1). The symbol used in the art for a gene coding for an aspartokinase polypeptide is lysC. In case the gene codes for a feedback resistant polypeptide variant the art typically uses symbols like lysC^{fbr} with fbr indicating feedback resistance.

SEQ ID NO:15 shows the nucleotide sequence of the coding sequence of the aspartokinase polypeptide of strain ATCC13032 and SEQ ID NO:16 the amino acid sequence of the encoded polypeptide. It is known in the art (see U.S. Pat. No. 6,893,848) that exchange of the amino acid Thr at position 311 of SEQ ID NO:16 for Ile imparts the enzyme a feedback resistance to inhibition by mixtures of L-lysine and L-threonine.

Accordingly it is preferred that the amino acid sequence of said feedback resistant aspartokinase polypeptide comprises the amino acid sequence of SEQ ID NO:16 containing isoleucine at position 311 instead of threonine.

Said amino exchange can be achieved by exchanging the nucleobase cytosine (c) at position 932 of SEQ ID NO:15 to give thymine (t). The acc codon for threonine is thus altered to the atc codon for isoleucine.

It is further known in the art that exchange of the gtg start codon of the coding sequence for the aspartokinase polypeptide for atg enhances expression of the polypeptide (e.g. WO201300827 A1 page 50, lines 23-31; US20090325244 A1; EP2796555 A2). Accordingly it is preferred that the sequence coding for a feedback resistant aspartokinase polypeptide begins with an atg start codon.

It was found that the bacteria modified according to the invention excreted L-amino acids selected from L-lysine, L-threonine and L-isoleucine, preferably L-lysine, into a suitable medium under suitable fermentation conditions in an increased manner with respect to one or more parameters selected from product concentration (i.e. amount of L-amino acid produced per volume, or mass unit resp., of medium/fermentation broth (e.g. g/l or g/kg)), product yield (i.e. amount of L-amino acid produced per carbon source consumed (e.g. g/g or kg/kg)), product formation rate (i.e. amount of L-amino acid produced per volume, or mass unit resp., of medium/fermentation broth and unit of time (e.g. g/l×h or g/kg×h)), and specific product formation rate (i.e. amount of L-amino acid produced per unit of time and mass unit of the producer (e.g. g/h×g dry mass)) as compared to the unmodified bacterium.

It is obvious that a higher product concentration facilitates product manufacturing e. g. purification and isolation. An increased product yield reduces the amount of raw material required. An increased product formation rate reduces the time required for a fermentation run thus increasing the availability of a given fermenter.

The bacteria used in the method according to the present invention thus contribute to the improvement of technical and economic aspects of the manufacturing of L-amino acids selected from L-lysine, L-threonine and L-isoleucine.

The term L-amino acids, where mentioned herein, in particular in the context of product formation, also comprises their ionic forms and salts, for example L-lysine mono hydrochloride or L-lysine sulfate in the case of the L-amino acid L-lysine.

For practicing the present invention bacteria of the genus Corynebacterium are used. A description of the genus Corynebacterium and the species comprised by this genus can be found in the article “Corynebacterium” by K. A. Bernard and G. Funke in Bergey's Manual of Systematics of Archaea and Bacteria (Bergey's Manual Trust, 2012).

Within the genus Corynebacterium the species Corynebacterium glutamicum is preferred.

Suitable strains of Corynebacterium glutamicum are wild strains of this species for example strains ATCC13032, ATCC14067 and ATCC13869, and L-amino acid excreting strains obtained from these wild strains, preferably L-amino acid excreting strains obtained from these wild strains.

Strain ATCC13032 (also available as DSM20300) is the taxonomic type strain of the species Corynebacterium glutamicum. Strain ATCC14067 (also available as DSM20411) is also known under the outdated designation Brevibacterium flavum. Strain ATCC13869 (also available as DSM1412) is also known under the outdated designation Brevibacterium lactofermentum. A taxonomic study of this group of bacteria based on DNA-DNA hybridization was done by Liebl et al. (International Journal of Systematic Bacteriology 41(2), 255-260, 1991). A comparative analysis of various strains of the species Corynebacterium glutamicum based on genome sequence analysis was provided by Yang and Yang (BMC Genomics 18(1):940).

A multitude of L-amino acid excreting strains of the genus Corynebacterium were obtained in the art during the past decades starting from strains such as ATCC13032, ATCC14067, ATCC13869 and the like. They were obtained as a result of strain development programs using inter alia methods like classical mutagenesis, selection for antimetabolite resistance as well as amplification and promotor modification of genes of the biosynthetic pathway of the L-amino acid in question by genetic engineering methods. Summaries may be found in L. Eggeling and M. Bott (Handbook of Corynebacterium glutamicum, CRC Press, 2005) or H. Yukawa and M. Inui (Corynebacterium glutamicum Biology and Biotechnolgy, Springer Verlag, 2013).

L-lysine excreting strains of the species Corynebacterium glutamicum are widely known in the art and can be used for the purpose of the present invention. For example Blombach et al (Applied and Environmental Microbiology 75(2), 419-427, 2009) describe strain DM1933, which is deposited under accession DSM25442 according to the Budapest treaty. Strain DM1933 was obtained from ATCC13032 by several steps of strain development. Other L-lysine excreting Corynebacterium glutamicum strains are e. g. described in WO2008033001 A1 and EP0841395 A1.

Summaries concerning the breeding of L-lysine excreting strains of Corynebacterium glutamicum may be found inter alia in L. Eggeling and M. Bott (Handbook of Corynebacterium glutamicum, CRC Press, 2005), V. F. Wendisch (Amino Acid Biosynthesis—Pathways, Regulation and Metabolic Engineering, Springer Verlag, 2007), H. Yukawa and M. Inui (Corynebacterium glutamicum Biology and Biotechnolgy, Springer Verlag, 2013), and Eggeling and Bott (Applied Microbiology and Biotechnology 99 (9), 338T-3394, 2015).

L-threonine excreting strains of the species Corynebacterium glutamicum are known in the art and can be used for the purpose of the present invention. For example EP0358940 A1 describes the strain DM368-2, which is deposited under DSM5399.

L-isoleucine excreting strains of the species Corynebacterium glutamicum are known in the art and can be used for the purpose of the present invention. For example U.S. Pat. No. 4,656,135 describes strain AJ12152, which is deposited under Ferm BP-760.

In case a wild strain, e.g. ATCC13032, ATCC13869 or ATCC14067, is in a first step subjected to the measures of the present invention the resulting strain is in a second step subjected to a strain development program targeted at the desired L-amino acid to obtain a bacterium according to the present invention.

The term DSM denotes the depository Deutsche Sammlung für Mikroorganismen and Zellkulturen located in Braunschweig, Germany. The term ATCC denotes the depository American Type Culure Collection located in Manasass, Va., US. The term FERM denotes the depository National Institute of Technology and Evaluation (NITE) located in Tokyo, Japan, Two other well-known depositories are KCCM and NRRL. The term KCCM denotes the depository Korean Culture Center of

Microorganisms located in Seoul, Korea. The term NRRL denotes the depository Agricultural Research Service Culture Collection located in Peoria, Ill., US.

Details regarding the biochemistry and chemical structure of polynucleotides and polypeptides as present in living things such as bacteria like Corynebacterium or Escherichia, for example, can be found inter alia in the text book “Biochemie” by Berg et al. (Spektrum Akademischer Verlag Heidelberg, Berlin, Germany, 2003; ISBN 3-8274-1303-6).

Polynucleotides consisting of deoxyribonucleotide monomers containing the nucleobases or bases resp. adenine (a), guanine (g), cytosine (c) and thymine (t) are referred to as deoxyribopolynucleotides or deoxyribonucleic acid (DNA). Polynucleotides consisting of ribonucleotide monomers containing the nucleobases or bases resp. adenine (a), guanine (g), cytosine (c) and uracil (u) are referred to as ribo-polynucleotides or ribonucleic acid (RNA). The monomers in said polynucleotides are covalently linked to one another by a 3′,5′-phosphodiester bond. By convention single strand polynucleotides are written from 5′- to 3′-direction. Accordingly a polynucleotide has a 5′-end and 3′-end. The order of the nucleotide monomers in the polynucleotide is commonly referred to as nucleotide sequence. Accordingly a polynucleotide is characterized by its nucleotide sequence. In bacteria, for example Corynebacterium or Escherichia, the DNA is typically present in double stranded form. Accordingly the length of a DNA molecule is typically given in base pairs (bp). The nucleotide sequence coding for a specific polypeptide is called coding sequence (cds).

A gene from a chemical point of view is a polynucleotide, usually a deoxyribopolynucleotide.

The term gene refers to a polynucleotide comprising a nucleotide sequence coding for a specific polypeptide (coding sequence) and the adjoining stop codon. In a broader sense the term includes regulatory sequences preceding and following the coding sequence. The preceding sequence is located at the 5′-end of the coding sequence and is also referred to as upstream sequence. A promotor is an example of a regulatory sequence located 5′ to the coding sequence. The sequence following the coding sequence is located at its 3′-end and also referred to as downstream sequence. A transcriptional terminator is an example of a regulatory sequence located 3′ to the coding sequence.

Polypeptides consist of L-amino acid monomers joined by peptide bonds. For abbreviation of L-amino acids the one letter code and three letter code of IUPAC (International Union of Pure and Applied Chemistry) is used. Due to the nature of polypeptide biosynthesis polypeptides have an amino terminal end and a carboxyl terminal end also referred to as N-terminal end and C-terminal end. The order of the L-amino acids or L-amino acid residues resp. in the polypeptide is commonly referred to as amino acid sequence. Polypeptides are also referred to as proteins.

Further it is known in the art that the start codon or initiation codon resp. gtg of a coding sequence as well as atg encodes the amino acid methionine.

Furthermore during the work for the present invention the amino acid sequences of the polypeptides encoded by the lmrB genes of different strains of the species Corynebacterium glutamicum were analyzed.

During the work for the present invention the coding sequence for the lmrB gene of Corynebacterium glutamicum strain DM1547, a strain obtained from strain ATCC13032, described in EP1239040 A2 (US2002119537 A1) and deposited as DSM13994, was analyzed and the amino acid sequence of the encoded polypeptide deduced. Said polypeptide was found to have the amino acid sequence of SEQ ID NO:8, wherein valine is contained at position 104. Its identity to the corresponding amino acid sequence from ATCC13032 is therefore 99.8%.

Accordingly said polypeptide conferring resistance to lincosamides, selected from lincomycin and clindamycin, preferably lincomycin, comprises an amino acid sequence being at least 90%, at least 95%, at least 96%, at least 97%, at least 98%, at least 99%, or 100% identical to the amino acid sequence of SEQ ID NO:8. In one specific embodiment, said polypeptide conferring resistance to lincosamides, selected from lincomycin and clindamycin, preferably lincomycin, comprises an amino acid sequence being at least 99.5%, preferably at least 99.8% identical to the amino acid sequence of SEQ ID NO:8, e.g. corresponding to an exchange of alanine by valine at position 104 of SEQ ID NO:8.

Said polypeptide is also referred to as LmrB polypeptide herewith.

Teachings for measuring resistance/sensitivity phenotypes can be found inter alia in text books of medical microbiology such as the textbook of H. Brandis, W. Köhler, H. J. Eggers and G. Pulverer (Lehrbuch der Medizinischen Mikrobiologie, 7^{th} edition, Gustav Fischer Verlag, 1994), by Swenson et al. in Manual of Clinical Microbiology (Murry et al. (eds), 1356-1367, American Society for Microbiology, 1995) or by Kim et al.

The amino acid sequence of said encoded LmrB polypeptide usually has a length of 481 amino acid residues. It is known in the art that the N-terminal amino acid methionine of an encoded polypeptide may be removed by an aminopeptidase during or after translation (Jocelyn E. Krebs, Elliott S. Goldstein and Stephan T. Kilpatrick: Lewin's Genes X. Jones and Bartlett Publishers, US, 2011).

The amino acid sequence of the encoded LmrB polypeptide from ATCC13032 shown in SEQ ID NO:2 is also shown in SEQ ID NO: 8. The nucleotide sequence encoding said polypeptide is shown in SEQ ID NO: 7 positions 1001 to 2443.

During the work for the present invention the coding sequence for the LmrB polypeptide of strain DM1547 described in EP1239040 A2 (US2002119537 A1) and deposited as DSM13994 was analyzed. Said coding sequence of strain DM1547 was found to be identical with that of strain ATCC13032 with the exception of position 311. Position 311 of the coding sequence corresponds to position 1311 of SEQ ID NO:7. Position 311 of the coding sequence in DM1547 contains the nucleobase thymine (t) resulting in a gtg codon for valine. Position 311 of the coding sequence in ATCC13032 contains cytosine (c) resulting in a gcg codon for alanine.

Hence the encoded LmrB polypeptide from DM1547 comprises the amino acid shown in SEQ ID NO:8, wherein valine is contained at position 104. The nucleotide sequence encoding said polypeptide is shown in SEQ ID NO: 7 positions 1001 to 2443, wherein thymine (I) is contained at position 1311.

The amino acid sequence of the encoded LmrB polypeptide from ATCC13869 shown in SEQ ID NO:4 is also shown in SEQ ID NO: 10. The nucleotide sequence encoding said polypeptide is shown in SEQ ID NO:9 positions 1001 to 2443.

The amino acid sequence of the encoded LmrB polypeptide from ATCC14067 shown in SEQ ID NO:6 is also shown in SEQ ID NO: 12. The nucleotide sequence encoding said polypeptide is shown in SEQ ID NO:11 positions 1001 to 2443.

During the work for the present invention it was shown that overexpression of the LmrB polypeptide of ATCC14067 in strain ATCC13032 and in the L-lysine producer DM1933 resulted in an increased resistance towards lincomycin.

The LmrB polypeptide comprises the amino acid sequence of SEQ ID NO:8, SEQ ID NO:8, wherein valine is contained at position 104, SEQ ID NO:10 or SEQ ID NO:12 with SEQ ID NO:8 and SEQ ID NO:8, wherein valine is contained at position 104, being particularly preferred. The nucleotide sequence encoding said LmrB polypeptide may also be selected from SEQ ID NO:7 positions 1001 to 2443, SEQ ID NO:7 positions 1001 to 2443, wherein thymine (t) is contained at position 1311, SEQ ID NO:9 positions 1001 to 2443 and SEQ ID NO:11 positions 1001 to 2443, with SEQ ID NO:7 positions 1001 to 2443 and SEQ ID NO:7 positions 1001 to 2443, wherein thymine (t) is contained at position 1311, being particularly preferred.

Teachings and information concerning the handling of and experimental work with polynucleotides may be found inter alia in the handbook of J. Sambrook et al. (Molecular Cloning, A Laboratory Manual, Cold Spring Harbor Laboratory Press, 1989), the textbook of C. R. Newton and A. Graham (PCR, Spektrum Akademischer Verlag, 1994) and the handbook of D. Rickwood and B. D. Hames (Gel electrophoresis of nucleic acids, a practical approach, IRL Press, 1982).

For sequence analysis of polynucleotides and polypeptides, e.g. sequence alignments, public software such as the CLC Genomics Workbench (Qiagen, Hilden, Germany) or the program MUSCLE provided by the European Bioinformatics Institute (EMBL-EBI, Hinxton, UK) may also be used.

During the work for the present invention it was found that modifying L-amino acid excreting bacteria of the genus Corynebacterium, preferably of the species Corynebacterium glutamicum, by eliminating the LmrB polypeptide increased their ability to excrete L-amino acids as compared to the unmodified bacterium, provided that the bacterium further comprises at least one copy of a polynucleotide coding for a feedback resistant aspartokinase as outlined before. The skilled artisan is aware of a number of methods of mutagenesis how to achieve said eliminating or switching off resp. of said LmrB polypeptide in the Corynebacterium.

In an embodiment of the present invention the modification of the lmrB polynucleotide resulting in an elimination of the LmrB activity is achieved by by
a) deleting one or two nucleotides in the part of the coding sequence of said lmrB polynucleotide corresponding to amino acids of positions 1 to 258 of the encoded amino acid sequence, or
b) deleting at least the part of the coding sequence of said LmrB polypeptide corresponding to amino acids of positions 210 to 258, positions 175 to 301, positions 119 to 364 or of positions 58 to 439 preferably of the encoded amino acid sequence according to SEQ ID:NO8, particularly preferred deleting at least the complete coding sequence.

As a consequence of said eliminating by deleting according to a) or b), preferably b), novel junction points or junction sites resp. are created in the chromosome of the Corynebacterium, preferably Corynebacterium glutamicum.

If for example the complete coding sequence is deleted a novel junction point is created in the chromosome of the Corynebacterium, preferably Corynebacterium glutamicum, which links the first nucleobase of the stop codon adjoining the coding sequence, e.g. the nucleobase at position 2444 of SEQ ID NO:7, SEQ ID NO:9 or SEQ ID NO:11, with the first nucleobase preceding the start codon of the coding sequence, e.g. the nucleobase at position 1000 of SEQ ID NO:7, SEQ ID NO:9 or SEQ ID NO:11.

Preferably, the modification of said lmrB polynucleotide results in an insertion of a recognition site for the restriction enzyme EcoRV in said polynucleotide. Thus, in a specific embodiment according to b) (above) the nucleotide sequence from positions 989 to 2455 of SEQ ID NO:7, of SEQ ID NO:7, wherein thymine (t) is contained at position 1311, of SEQ ID NO:9 or of SEQ ID NO:11, which comprises the coding sequence of said LmrB polypeptide and the adjoining stop codon, is deleted and the nucleotide sequence gatatc, which is the recognition site for the restriction endonuclease inserted into the site of deletion.

Thus a novel junction site is created in the chromosome of the Corynebacterium glutamicum characterized by the gatatc nucleotide sequence bridge between the nucleobase at position 988 and the nucleobase at position 2456 of SEQ ID NO:7, SEQ ID NO:9 or SEQ ID NO:11. Strains ATCC13032, DM1547 and ATCC13869 contain at position 988 the nucleobase cytosine (c) as shown in SEQ ID NO:7 and SEQ ID NO:9. Strain ATCC14067 contains at position 988 the nucleobase thymine (t) as shown in SEQ ID NO:11. At position 2456 the three strains contain the nucleobase thymine as shown in SEQ ID NO:7, SEQ ID NO:9 and SEQ ID NO:11.

The nucleotide sequence of the novel junction site created including the nucleotide sequences upstream and downstream therefrom are shown in SEQ ID NO:13 and SEQ ID NO:14 and table 2, table 3, table 4, table 5, table 6, table 7 and table 8.

Accordingly, in a more specific embodiment of the present invention a deletion in the chromosome of a Corynebacterium glutamicum, preferably ATCC13032, ATCC13869, ATCC14067 and L-amino acid excreting strains obtained from these strains, comprising the complete coding sequence for the LmrB polypeptide and the adjoining stop codon accompanied by the insertion of the recognition site for the restriction enzyme EcoRV is identified by a nucleotide sequence selected from table 2.

Accordingly in another more specific embodiment of the present invention a deletion in the chromosome of Corynebacterium glutamicum strains, preferably ATCC13032, ATCC13869 and L-amino acid excreting strains obtained from these strains, comprising the complete coding sequence for the LmrB polypeptide and the adjoining stop codon accompanied by the insertion of the recognition site for the restriction enzyme EcoRV is identified by a nucleotide sequence selected from table 3.

Accordingly in another more specific embodiment of the present invention a deletion in the chromosome of Corynebacterium glutamicum strains, preferably ATCC13032, ATCC13869 and L-amino acid excreting strains obtained from these strains, comprising the complete coding sequence for the LmrB polypeptide and the adjoining stop codon accompanied by the insertion of the recognition site for the restriction enzyme EcoRV is identified by a nucleotide sequence selected from table 4.

Accordingly in another more specific embodiment of the present invention a deletion in the chromosome of Corynebacterium glutamicum strains, preferably ATCC13032, ATCC13869 and L-amino acid excreting strains obtained from these strains, comprising the complete coding sequence for the LmrB polypeptide and the adjoining stop codon accompanied by the insertion of the recognition site for the restriction enzyme EcoRV is identified by a nucleotide sequence selected from table 5.

Accordingly in another more specific embodiment of the present invention a deletion in the chromosome of Corynebacterium glutamicum strains, preferably ATCC13032, ATCC13869 and L-amino acid excreting strains obtained from these strains, comprising the complete coding sequence for the LmrB polypeptide and the adjoining stop codon accompanied by the insertion of the recognition site for the restriction enzyme EcoRV is identified by a nucleotide sequence selected from table 6.

Accordingly in another more specific embodiment of the present invention a deletion in the chromosome of Corynebacterium glutamicum strains, preferably ATCC14067 and L-amino acid excreting strains obtained therefrom, comprising the complete coding sequence for the LmrB polypeptide and the adjoining stop codon accompanied by the insertion of the recognition site for the restriction enzyme EcoRV is identified by a nucleotide sequence selected from table 7.

Accordingly in another more specific embodiment of the present invention a deletion in the chromosome of Corynebacterium glutamicum strains, preferably ATCC14067 and L-amino acid excreting strains obtained therefrom, comprising the complete coding sequence for the LmrB polypeptide and the adjoining stop codon accompanied by the insertion of the recognition site for the restriction enzyme EcoRV is identified by a nucleotide sequence selected from table 8.

TABLE 2
List of nucleotide sequences indicating a deletion in the chromosome
of a Corynebacterium glutamicum comprising the coding sequence
and the adjoining stop codon of the ImrB gene in accordance with the
present invention. The nucleotide sequence for the recognition site for
the restriction endonuclease EcoRV extends from position 789 to
794 in SEQ ID NO: 13 and SEQ ID NO: 14.

| line | nucleotide sequence | length* |
|---|---|---|
| a | SEQ ID NO: 13 positions 789 to 801 | 13 |
| b | SEQ ID NO: 13 positions 789 to 802 | 14 |
| c | SEQ ID NO: 13 positions 789 to 803 | 15 |
| d | SEQ ID NO: 13 positions 789 to 804 | 16 |
| e | SEQ ID NO: 13 positions 789 to 805 | 17 |

*length in nucleobases or base pairs resp.

TABLE 3
List of nucleotide sequences indicating a deletion in the chromosome
of strains of Corynebacterium glutamicum comprising the coding
sequence and the adjoining stop codon of the lmrB gene in accordance
with the present invention. The nucleotide sequence of the recognition
site for the restriction endonuclease EcoRV extends from position
789 to 794 in SEQ ID NO: 13.

| line | nucleotide sequence | length* |
|---|---|---|
| a | SEQ ID NO: 13 positions 788 to 800 | 13 |
| b | SEQ ID NO: 13 positions 788 to 801 | 14 |
| c | SEQ ID NO: 13 positions 788 to 802 | 15 |
| d | SEQ ID NO: 13 positions 788 to 803 | 16 |
| e | SEQ ID NO: 13 positions 788 to 804 | 17 |

*length in nucleobases or base pairs resp.

TABLE 4
List of nucleotide sequences indicating a deletion in the chromosome
of strains of Corynebacterium glutamicum comprising the coding
sequence and the adjoining stop codon of the lmrB gene in accordance
with the present invention. The nucleotide sequence of the recognition
site for the restriction endonuclease EcoRV extends from position
789 to 794 in SEQ ID NO: 13.

| line | nucleotide sequence | length* |
|---|---|---|
| a | SEQ ID NO: 13 positions 787 to 798 | 12 |
| b | SEQ ID NO: 13 positions 787 to 899 | 13 |
| c | SEQ ID NO: 13 positions 787 to 800 | 14 |
| d | SEQ ID NO: 13 positions 787 to 801 | 15 |
| e | SEQ ID NO: 13 positions 787 to 802 | 16 |
| f | SEQ ID NO: 13 positions 787 to 803 | 17 |

*length in nucleobases or base pairs resp.

TABLE 5
List of nucleotide sequences indicating a deletion in the chromosome
of strains of Corynebacterium glutamicum comprising the coding
sequence and the adjoining stop codon of the lmrB gene in accordance
with the present invention. The nucleotide sequence of the recognition
site for the restriction endonuclease EcoRV extends from position
789 to 794 in SEQ ID NO: 13.

| line | nucleotide sequence | length* |
|---|---|---|
| a | SEQ ID NO: 13 positions 784 to 795 | 12 |
| b | SEQ ID NO: 13 positions 783 to 795 | 13 |
| c | SEQ ID NO: 13 positions 782 to 795 | 14 |
| d | SEQ ID NO: 13 positions 781 to 795 | 15 |
| e | SEQ ID NO: 13 positions 780 to 795 | 16 |
| f | SEQ ID NO: 13 positions 779 to 795 | 17 |

*length in nucleobases or base pairs resp.

TABLE 6
List of nucleotide sequences indicating a deletion in the chromosome
of strains of Corynebacterium glutamicum comprising the coding
sequence and the adjoining stop codon of the lmrB gene in accordance
with the present invention. The nucleotide sequence of the recognition
site for the restriction endonuclease EcoRV extends from position
789 to 794 in SEQ ID NO: 13.

| line | nucleotide sequence | length* |
|---|---|---|
| a | SEQ ID NO: 13 positions 784 to 796 | 13 |
| b | SEQ ID NO: 13 positions 783 to 796 | 14 |
| c | SEQ ID NO: 13 positions 782 to 796 | 15 |
| d | SEQ ID NO: 13 positions 781 to 796 | 16 |
| e | SEQ ID NO: 13 positions 780 to 796 | 17 |

*length in nucleobases or base pairs resp.

TABLE 7
List of nucleotide sequences indicating a deletion in the chromosome
of strains of Corynebacterium glutamicum comprising the coding
sequence and the adjoining stop codon of the lmrB gene in accordance
with the present invention. The nucleotide sequence of the recognition
site for the restriction endonuclease EcoRV extends from position
789 to 794 in SEQ ID NO: 14.

| line | nucleotide sequence | length* |
|---|---|---|
| a | SEQ ID NO: 14 positions 782 to 795 | 14 |
| b | SEQ ID NO: 14 positions 781 to 795 | 15 |
| c | SEQ ID NO: 14 positions 780 to 795 | 16 |
| d | SEQ ID NO: 14 positions 779 to 795 | 17 |
| e | SEQ ID NO: 14 positions 778 to 795 | 18 |

*length in nucleobases or base pairs resp.

TABLE 8
List of nucleotide sequences indicating a deletion in the chromosome
of strains of Corynebacterium glutamicum comprising the coding
sequence and the adjoining stop codon of the lmrB gene in accordance
with the present invention. The nucleotide sequence of the recognition
site for the restriction endonuclease EcoRV extends from position
789 to 794 in SEQ ID NO: 14.

| line | nucleotide sequence | length* |
|---|---|---|
| a | SEQ ID NO: 14 positions 788 to 801 | 14 |
| b | SEQ ID NO: 14 positions 788 to 802 | 15 |
| c | SEQ ID NO: 14 positions 788 to 803 | 16 |
| d | SEQ ID NO: 14 positions 788 to 804 | 17 |

*length in nucleobases or base pairs resp.

In another embodiment said modification of the lrmB polynucleotide resulting in an elimination of the LmrB activity is achieved by

a) inserting one or two nucleotides into the part of the coding sequence of said LmrB polypeptide corresponding to amino acids of positions 1 to 258 of the encoded amino acid sequence,

b) inserting a gene coding for a polypeptide of the biosynthetic pathway of an L-amino acid selected from L-lysine, L-threonine and L-isoleucine, preferably L-lysine, into the part of the coding sequence corresponding to amino acids of positions 175 to 258, preferably positions 210 to 258 of the amino acid sequence of said LmrB polypeptide.

Said genes coding for a polypeptide of the biosynthetic pathway of L-lysine is preferably selected from the list comprising
a gene pyc of Corynebacterium glutamicum coding for pyruvate carboxylase (EC 6.4.1.1) as e.g. described in WO1999018228 A2 (U.S. Pat. No. 7,267,967 B1) or EP2107128 A2,
a gene aspB of Corynebacterium glutamicum coding for aspartate amino transferase (EC 2.6.1.1) as e.g. described in EP0219027 A2 or WO2008033001 A1,
a gene lysC of Corynebacterium glutamicum coding for an aspartate kinase, preferably feedback resistant aspartate kinase (EC 2.7.2.4), as e.g. described in U.S. Pat. No. 6,893,848,
a gene asd of Corynebacterium glutamicum coding for aspartate semialdehyde dehydrogenase (EC 1.2.1.11) as e.g. described in EP0387527 A1 or WO2008033001 A1,
a gene dapA of Corynebacterium glutamicum coding for dihydrodipicolinate synthase (EC 4.3.3.7) as e.g. described in EP0197335 A1,
a gene dapB of Corynebacterium glutamicum coding for dihydrodipicolinate reductase (EC 1.17.1.8) as e.g. described in U.S. Pat. No. 8,637,295 or EP0841395 A1,
a gene ddh of Corynebacterium glutamicum coding for diaminopimelate dehydrogenase (EC 1.4.1.16) as e.g. described in EP0811682 A2,
a gene lysE of Corynebacterium glutamicum coding for lysine permease as e.g. described in WO9723597 A2 (U.S. Pat. No. 6,858,406 B1),
a gene dapF of Corynebacterium glutamicum coding for diaminopimelate epimerase as e.g. described in U.S. Pat. No. 6,670,156, and
a gene lysA of Corynebacterium glutamicum coding for diaminopimelate decarboxylase (EC 4.1.1.20) as e.g. described in EP0811682 A2.

In a further embodiment said modification of the lrmB polynucleotide resulting in an elimination of the LmrB activity is achieved by substituting one or more codons coding for an amino acid of said polypeptide with a taa-, tga- or tag-stop codon, preferably a taa- or tga-stop codon, particularly preferred taa-stop codon, in the part of coding sequence corresponding to amino acids of positions 1 to 258, preferably positions 1 to 20, particularly preferred positions 1 to 2 of the encoded amino acid sequence, e.g. of the amino acid sequence according to SEQ ID NO: 8.

In the expression “one or more codons” the term “more” means any positive integer or natural number resp. up to 258, preferably up to 20, particularly preferred up to 10, more particularly preferred up to 3 or 2, when it relates to a substitution of a codon for an amino acid in the part of coding sequence corresponding to amino acids of positions 1 to 258 of the encoded amino acid sequence.

It means any positive integer or natural number resp. up to 20, preferably up to 10, particularly preferred up to 3 or 2, when it relates to a substitution of a codon for an amino acid in the part of coding sequence corresponding to amino acids of positions 1 to 20 of the encoded amino acid sequence.

It means 2 (two) when it relates to a substitution of a codon for an amino acid in the part of coding sequence corresponding to amino acids of positions 1 to 2 of the encoded amino acid sequence.

In a further embodiment said modification of the lrmB polynucleotide resulting in an elimination of the LmrB activity is achieved by inserting at least one stop codon, preferably no more than 10 stop codons, selected from taa-, tga- or tag-stop codon, preferably taa- or tga-stop codon, particularly preferred taa-stop codon, between at least one pair of adjacent or neighboring codons in the part of coding sequence corresponding to amino acids of positions 1 to 258, preferably positions 1 to 20, particularly preferred positions 1 to 2 of the encoded amino acid sequence of said polypeptide.

For example one or two taa-stop codon(s) may be inserted between the codons coding for the amino acids at position 1 and 2 of the amino acid sequence shown in SEQ ID NO:8 or SEQ ID NO:8, wherein valine is contained at position 104, or accordingly between positions 1003 and 1004 of the nucleotide sequence shown in SEQ ID NO:7 or of the nucleotide sequence shown in SEQ ID NO:7, wherein thymine (t) is contained at position 1311.

A common method of mutating genes of Corynebacterium glutamicum is the method and of gene replacement described by Schwarzer and Pühler (Bio/Technology 9, 84-87 (1991)) and further elaborated by Schäfer et al. (Gene 145, 69-73 (1994)).

Peters-Wendisch et al. (Microbiology 144, 915-927 (1998)) used the gene replacement method to inactivate the pyc gene of Corynebacterium glutamicum encoding pyruvate carboxylase. Schäfer et al, used the method to incorporate a deletion into the horn-thrB gene region of Corynebacterium glutamicum. In EP1094111 the method was used to incorporate a deletion into the pck gene of Corynebacterium glutamicum encoding phosphoenol pyruvate carboxykinase.

In the gene replacement method, a mutation, such as, for example, a deletion, insertion or substitution of at least one nucleobase, is constructed in vitro in the nucleotide sequence of the gene in question. In the context of the present invention the nucleotide sequence of the gene in question (lmrB gene) encodes a polypeptide having the activity of conferring resistance to lincosamides as specified in this invention. Examples of such nucleotide sequences are SEQ ID NO:7, SEQ ID NO:7 containing thymine (t) at position 1311, SEQ ID NO:9 and SEQ ID NO:11 of the sequence listing. In the context of the present invention the mutation is a deletion, insertion or substitution of at least one nucleobase, preferably deletion of at least one nucleobase, located in the coding sequence of said lmrB gene.

The mutated nucleotide sequence of the gene in question comprises i) a nucleotide sequence at the 5′-end of the site of mutation, which is also referred to as 5′-flanking sequence or upstream sequence in the art, ii) a nucleotide sequence at the 3′-end of the site of mutation, which is also referred to as 3′-flanking sequence or downstream sequence in the art, and iii) the nucleotide sequence of the site of mutation between i) and ii). In case of a deletion the site of mutation is besides the lack of a specific sequence, also characterized by the flanking sequences forming the novel function point. In the context of the present invention said deletion preferably concerns the complete coding sequence for the LmrB polypeptide or a part thereof.

For some applications it may be convenient to further incorporate a suitable polynucleotide into said site of mutation. Said suitable polynucleotide may inter alia contain the coding sequence for an enzyme of the biosynthetic pathway of an L-amino acid, e. g. the coding sequence for the enzyme aspartokinase, which is an enzyme of the L-lysine biosynthetic pathway, or the nucleotide sequence of the recognition site for a restriction enzyme useful for further strain improvement.

An example of a mutated nucleotide sequence in the context of the present invention is shown in SEQ ID NO:13. The 5′-flanking sequence consists of the nucleotide sequence from positions 201 to 988 of SEQ ID NO:7. The 3′-flanking sequence consists of the nucleotide sequence from positions 2456 to 3246 of SEQ ID NO:7. The nucleotide sequence from positions 989 to 2455 of SEQ ID NO:7 comprising the coding sequence of the LmrB polypeptide was removed or deleted resp., and additionally the nucleotide sequence of the recognition site for the restriction endonuclease EcoRV incorporated as shown from positions 789 to 794 of SEQ ID NO:13.

Another example of a mutated nucleotide sequence in the context of the present invention is shown in SEQ ID NO:14. The 5′-flanking sequence consists of the nucleotide sequence from positions 201 to 988 of SEQ ID NO:11. The 3′-flanking sequence consists of the nucleotide sequence from positions 2456 to 3246 of SEQ ID NO:11. The nucleotide sequence from positions 989 to 2455 of SEQ ID NO:11 comprising the coding sequence of the LmrB polypeptide was removed or deleted resp. and additionally the nucleotide sequence of the recognition site for the restriction endonuclease EcoRV incorporated as shown from positions 789 to 794 of SEQ ID NO:14.

The mutated nucleotide sequence constructed is cloned into a plasmid vector that is not capable of autonomous replication in Corynebacterium glutamicum. Said plasmid vector comprising said mutated nucleotide sequence is subsequently transferred into the desired strain of Corynebacterium glutamicum by transformation or conjugation. After two events of homologous recombination comprising a recombination event within the 5′-flanking sequence provided by the plasmid vector with the homologous sequence of the Corynebacterium glutamicum chromosome and a recombination event within the 3′-flanking sequence provided by the plasmid vector with the homologous sequence of the Corynebacterium glutamicum chromosome, one effecting integration and one effecting excision of said plasmid vector, the mutation is incorporated in the Corynebacterium glutamicum chromosome. Thus the nucleotide sequence of the gene in question contained in the chromosome of said desired strain is replaced by the mutated nucleotide sequence.

An event of homologous recombination may also be referred to as crossing over.

It is preferred that the L-amino acid excreting strains of Corynebacterium, preferably Corynebacterium glutamicum, of the present invention have the ability to excrete ≥0.1 g/l, preferably ≥0.25 g/l, particularly preferred ≥0.5 g/l of the desired L-amino acid in a suitable medium under suitable conditions.

In a fermentative process according to the invention a Corynebacterium, preferably Corynebacterium glutamicum, modified in accordance with the present invention and having the ability to excrete an L-amino is cultivated in a suitable medium under suitable conditions. Due to said ability to excrete said L-amino acid the concentration of the L-amino acid increases and accumulates in the medium during the fermentative process and the L-amino acid is thus produced.

The fermentative process may be discontinuous process like a batch process or a fed batch process or a continuous process. A summary concerning the general nature of fermentation processes is available in the textbook by H. Chimiel (Bioprozesstechnik, Spektrum Akademischer Verlag, 2011), in the textbook of C. Ratledge and B. Kristiansen (Basic Biotechnology, Cambridge University Press, 2006) or in the textbook of V. C. Hass and R. Pörtner (Praxis der Bioprozesstechnik Spektrum Akademischer Verlag, 2011).

A suitable medium used for the production of an L-amino acid by a fermentative process contains a carbon source, a nitrogen source, a phosphorus source, inorganic ions and other organic compounds as required.

Suitable carbon sources include glucose, fructose, sucrose as well as the corresponding raw materials like starch hydrolysate, molasses or high fructose corn syrup.

As nitrogen source organic nitrogen-containing compounds such as peptones, meat extract, soy bean hydrolysates or urea, or inorganic compounds such as ammonium sulphate, ammonium chloride, ammonium phosphate, ammonium carbonate, ammonium nitrate, ammonium gas or aqueous ammonia can be used.

As phosphorus source, phosphoric acid, potassium dihydrogen phosphate or dipotassium hydrogen phosphate or the corresponding sodium-containing salts can be used.

Inorganic ions like potassium, sodium, magnesium, calcium, iron and further trace elements etc. are supplied as salts of sulfuric acid, phosphoric acid or hydrochloric acid.

Other organic compounds means essential growth factors like vitamins e. g. thiamine or biotin or L-amino acids e. g. L-homoserine.

The media components may be added to the culture in form of a single batch or be fed in during the cultivation in a suitable manner.

During the fermentative process the pH of the culture can be controlled by employing basic compounds such as sodium hydroxide, potassium hydroxide, ammonia or aqueous ammonia, or acidic compounds such as phosphoric acid or sulphuric acid in a suitable manner. The pH is generally adjusted to a value of from 6.0 to 8.5, preferably 6.5 to 8.0. To control foaming, it is possible to employ antifoam agents such as, for example, fatty acid polyglycol esters. To maintain the stability of plasmids, it is possible to add to the medium suitable selective substances such as, for example, antibiotics. The fermentative process is preferably carried out under aerobic conditions. In order to maintain these conditions, oxygen or oxygen-containing gas mixtures such as, for example air are introduced into the culture. The fermentative process is carried out, where appropriate, at elevated pressure, for example at an elevated pressure of 0.03 to 0.2 MPa. The temperature of the culture is normally from 25° C. to 40° C., preferably from 30° C. to 37° C. In a discontinuous process, the cultivation is continued until an amount of the desired L-amino acid sufficient for being recovered has been formed. The cultivation is then completed. This aim is normally achieved within 10 hours to 160 hours. In continuous processes, longer cultivation times are possible.

Examples of suitable media and culture conditions can be found inter alia in L. Eggeling and M. Bott (Handbook of Corynebacterium glutamicum, CRC Press, 2005) and the patent documents U.S. Pat. Nos. 5,770,409, 5,990,350, 5,275,940, 5,763,230 and 6,025,169.

Thus the fermentative process results in a fermentation broth which contains the desired L-amino acid. A product containing the L-amino acid is then recovered in liquid or solid from the fermentation broth. A “fermentation broth” means a medium in which a Corynebacterium of the invention has been cultivated for a certain time and under certain conditions.

When the fermentative process is completed, the resulting fermentation broth accordingly comprises:

a) the biomass (cell mass) of the Corynebacterium of the invention, said biomass having been produced due to propagation of the cells of said Corynebacterium,

b) the desired fine chemical accumulated during the fermentative process,

c) the organic by-products accumulated during the fermentative process, and

d) the components of the medium employed which have not been consumed in the fermentative process.

The organic by-products include compounds which may be formed by the Corynebacterium of the invention during the fermentative process in addition to production of the desired L-amino acid.

The fermentation broth is removed from the culture vessel or fermentation tank, collected where appropriate, and used for providing a product containing the fine chemical, preferably an L-amino acid-containing product, in liquid or solid form. The expression “recovering the fine chemical-containing product” is also used for this. In the simplest case, the L-amino acid-containing fermentation broth itself, which has been removed from the fermentation tank, constitutes the recovered product.

The fermentation broth can subsequently be subjected to one or more of the measures selected from the group consisting of:

a) partial (>0% to <80%) to complete (100%) or virtually complete (≥80%, ≥90%, ≥95%, ≥96%, ≥97%, ≥98%, ≥99%) removal of the water,

b) partial (>0% to <80%) to complete (100%) or virtually complete (≥80%, ≥90%, ≥95%, ≥96%, ≥97%, ≥98%, ≥99%) removal of the biomass, the latter being optionally inactivated before removal,

c) partial (>0% to <80%) to complete (100%) or virtually complete (≥80%, ≥90%, ≥95%, ≥96%, ≥97%, ≥98%, ≥99%, ≥99.3%, ≥99.7%) removal of the organic by-products formed during the fermentative process, and

d) partial (>0%) to complete (100%) or virtually complete (24 80%, ≥90%, ≥95%, ≥96%, ≥97%, ≥98%, ≥99%, ≥99.3%, ≥99.7%) removal of the residual components of the medium employed or of the residual input materials resp., which have not been consumed in the fermentative process.

An abundance of technical instructions for measures a), b), c) and d) are available in the art.

Removal of water (measure a)) can be achieved inter alia by evaporation, using e.g. a falling film evaporator, by reverse osmosis or nanofiltration. The concentrates thus obtained can be further worked up by spray drying or spray granulation. It is likewise possible to dry the fermentation broth directly using spray drying or spray granulation.

Removal of the biomass (measure b)) can be achieved inter alia by centrifugation, filtration or decantation or a combination thereof.

Removal of the organic by-products (measure c)) or removal of residual components of the medium (measure d) can be achieved inter alia by chromatography, e.g. ion exchange chromatography, treatment with activated carbon or crystallization. In case the organic by-products or residual components of the medium are present in the fermentation broth as solids they can be removed by measure b).

General instructions on separation, purification and granulation methods can be found inter alia in the book of R. Ghosh “Principles of Bioseperation Engineering” (World Scientific Publishing, 2006), the book of F. J. Dechow “Seperation and Purification Techniques in Biotechnology” (Noyes Publications, 1989), the article “Bioseparation” of Shaeiwitz et al. (Ullmann's Encyclopedia of Industrial Chemistry, Wiley-VCH, 2012) and the book of P. Semo et al. “Granulieren” (Editio Cantor Verlag, 2007).

A downstream processing scheme for L-lysine products can be found in the article “L-lysine Production” of R. Kelle et al. (L. Eggeling and M. Bott (Handbook of Corynebacterium glutamicum, CRC Press, 2005)). U.S. Pat. No. 5,279,744 teaches the manufacturing of a purified L-lysine product by ion exchange chromatography. U.S. Pat. No. 5,431,933 teaches the manufacturing of dry L-amino acid products, e. g. an L-lysine product, containing most of the constituents of the fermentation broth.

Thus a concentration or purification of the desired L-amino acid is achieved and a product having the desired content of said L-amino acid is provided.

Analysis of L-amino acids to determine the concentration at one or more time(s) during the fermentation can take place by separating the L-amino acids by means of ion exchange chromatography, preferably cation exchange chromatography, with subsequent post-column derivatization using ninhydrin, as described in Spackman et al, (Analytical Chemistry 30: 1190-1206 (1958)). It is also possible to employ ortho-phthalaldehyde rather than ninhydrin for post-column derivatization. An overview article on ion exchange chromatography can be found in Pickering (LC.GC (Magazine of Chromatographic Science 7(6):484-487 (1989)). It is likewise possible to carry out a pre-column derivatization, for example using ortho-phthalaldehyde or phenyl isothiocyanate, and to fractionate the resulting amino acid derivates by reversed-phase chromatography (RP), preferably in the form of high-performance liquid chromatography (HPLC). A method of this type is described, for example, in Lindroth et al. (Analytical Chemistry 51:1167-1174 (1979)). Detection is carried out photometrically (absorption, fluorescence). A review regarding amino acid analysis can be found inter alia in the textbook “Bioanalytik” by Lottspeich and Zorbas (Spektrum Akademischer Verlag, Heidelberg, Germany 1998).

Having generally described this invention, a further understanding can be obtained by reference to certain specific examples which are provided herein for purposes of illustration only, and are not intended to be limiting unless otherwise specified.

### EXPERIMENTAL SECTION

### A) Materials and Methods

The molecular biology kits, primers and chemicals used and some details of the methods applied are briefly described herewith.

1. Chemicals

a. Kanamycin solution from Streptomyces kanamyceticus was purchased from Sigma Aldrich (St. Louis, USA, Cat. no. K0254).

b. Nalidixic acid sodium salt was purchased from Sigma Aldrich (St. Louis, USA, Cat. no. N4382).

c. Lincomycin-hydrochlorid was purchased from Sigma Aldrich (St. Louis, USA, Cat. no. 62143-1g).

d. IPTG (Isopropyl β-D-1-thiogalactopyranoside) was purchased from Carl-Roth (Karlsruhe, Germany, Cat. no. 2316.4.)

e. If not stated otherwise, all other chemicals were purchased analytically pure from Merck (Darmstadt, Germany), Sigma Aldrich (St. Louis, USA) or Carl-Roth (Karlsruhe, Germany).

2. Cultivation

If not stated otherwise, all cultivation/incubation procedures were performed as follows herewith;

a. LB broth (MILLER) from Merck (Darmstadt, Germany; Cat. no. 110285) was used to cultivate E. coli strains in liquid medium. The liquid cultures (10 ml liquid medium per 100 ml Erlenmeyer flask with 3 baffles) were incubated in the Infors HT Multitron standard incubator shaker from Infors GmbH (Bottmingen, Switzerland) at 37° C. and 200 rpm.

b. LB agar (MILLER) from Merck (Darmstadt, Germany Cat. no. 110283) was used for cultivation of E. coli strains on agar plates. The agar plates were incubated at 37° C. in an INCU-Line® mini incubator from VWR (Radnor, USA).

c. Brain heart infusion broth (BHI) from Merck (Darmstadt, Germany; Cat. no. 110493) was used to cultivate C. glutamicum strains in liquid medium. The liquid cultures (10 ml liquid medium per 100 ml Erlenmeyer flask with 3 baffles) were incubated in the Infors HT Multitron standard incubator shaker from Infors GmbH (Bottmingen, Switzerland) at 33°C. and 200 rpm.

d. Brain heart agar (BHI-agar) from Merck (Darmstadt, Germany; Cat. no. 113825) was used for cultivation of C. glutamicum strains on agar plates. The agar plates were incubated at 33° C. in an incubator from Heraeus Instruments with Kelvitron® temperature controller (Hanau, Germany).

3. Determining Optical Density

a. The optical density of bacterial suspensions in shake flask cultures was determined at 600 nm (OD600) using the BioPhotometer from Eppendorf AG (Hamburg, Germany).

b. The optical density of bacterial suspensions produced in the Wouter Duetz (WDS) micro fermentation system (24-Well Plates) was determined at 660 nm (OD660) with the GENios™ plate reader from Tecan Group AG (Männedorf, Switzerland).

4. Centrifugation

a. Benchtop centrifuge for reaction tubes with a volume up to 2 ml

Bacterial suspensions with a maximum volume of 2 ml were caused to sediment using 1 ml or 2 ml reaction tubes (e.g. Eppendorf Tubes® 3810X) using an Eppendorf 5417 R centrifuge (5 min. at 13.000 rpm).

b. Benchtop centrifuge for tubes with a volume up to 50 ml

Bacterial suspensions with a maximum volume of 50 ml were caused to sediment using 15 ml or 50 ml centrifuge tubes (e.g. Falcon™ 50 ml Conical Centrifuge Tubes) using an Eppendorf 5810 R centrifuge for 10 min, at 4.000 rpm.

5. DNA Isolation

a. Plasmid DNA was isolated from E. coli cells using the QIAprep Spin Miniprep Kit from Qiagen (Hilden, Germany, Cat. No. 27106) according to the instructions of the manufacturer.

b. Total DNA from C. glutamicum was isolated using the method of Eikmanns et al. (Microbiology 140, 1817-1828, 1994).

6. Polymerase Chain Reaction (PCR)

PCR with a proof reading (high fidelity) polymerase was used to amplify a desired segment of DNA before Gibson Assembly or Sanger sequencing.

Non-proof reading polymerase Kits were used for determining the presence or absence of a desired DNA fragment directly from E. coli or C. glutamicum colonies.

a. The Phusion® High-Fidelity DNA Polymerase Kit (Phusion Kit) from New England BioLabs Inc. (Ipswich, USA; Cat. No. M0530) was used for template-correct amplification of selected DNA regions according to the instructions of the manufacturer (see table 9).

TABLE 9
Thermocycling conditions for PCR with Phusion ®
High-Fidelity DNA Polymerase Kit from NEB Inc.
PCR-program

| | Time | T | |
|---|---|---|---|
| Step | [min.:sec.] | [° C.] | Description |
| 1 | 00:30 | 98 | Initial denaturation step |
| 2 | 00:05 | 98 | Denaturation step |
| 3 | 00:30 | 60 | Annealing step |
| 4 | 00:xx | 72 | Elongation step |
| | | | 1 min. per kb DNA |
| | | | Repeat step 2 to 4: 35 x |
| 5 | 05:00 | 72 | Final elongation step |
| 6 | Hold | 4 | Cooling step |

b. Taq PCR Core Kit (Taq Kit) from Qiagen (Hilden, Germany; Cat. No. 201203) was used to amplify a desired segment of DNA in order to confirm its presence. The kit was used according to the instructions of the manufacturer (see table 10).

TABLE 10
Thermocycling conditions for PCR with Taq
PCR Core Kit (Taq Kit) from Qiagen.
PCR-program

| | Time | T | |
|---|---|---|---|
| Step | [min.:sec.] | [° C.] | Description |
| 1 | 05:00 | 94 | Initial denaturation step |
| 2 | 00:30 | 94 | Denaturation step |
| 3 | 00:30 | 52 | Annealing step |
| 4 | 01:20 | 72 | Elongation step |
| | | | 1 min. per kb DNA |
| | | | Repeat step 2 to 4: 35 x |
| 5 | 04:00 | 72 | Final elongation step |
| 6 | Hold | 4 | Cooling step |

c. SapphireAmp® Fast PCR Master Mix (Sapphire Mix) from Takara Bio Inc (Takara Bio Europe S.A.S., Saint-Germain-en-Laye, France; Cat. No. RR350A/B) was used as an alternative to confirm the presence of a desired segment of DNA in cells taken from E. coli or C. glutamicum colonies according to the instructions of the manufacturer (see table 11).

TABLE 11
Thermocycling conditions for PCR with SapphireAmp ®
Fast PCR Master Mix (Sapphire Mix) from Takara Bio Inc.
PCR-program

| | Time | T | |
|---|---|---|---|
| Step | [min.:sec.] | [° C.] | Description |
| 1 | 01:00 | 94 | Initial denaturation step |
| 2 | 00:05 | 98 | Denaturation step |
| 3 | 00:05 | 55 | Annealing step |
| 4 | 00:05 | 72 | Elongation step |
| | | | Repeat step 2 to 4: 30 x |
| 5 | 04:00 | 72 | Final elongation step |
| 6 | Hold | 4 | Cooling step |

d. Primer

The oligonucleotides used were synthesized by eurofins genomics GmbH (Ebersberg, Germany) using the phosphoramidite method described by McBride and Caruthers (Tetrahedron Lett. 24, 245-248, 1983).

e. Template

As PCR template either a suitably diluted solution of isolated plasmid DNA or of total DNA isolated from a C. glutamicum liquid culture or the total DNA contained in a colony (colony PCR) was used. For said colony PCR the template was prepared by taking cell material with a toothpick from a colony on an agar plate and placing the cell material directly into the PCR reaction tube. The cell material was heated for 10 sec. with 800 W in a microwave oven type Mikrowave & Grill from SEVERIN Elektrogeräte GmbH (Sundern, Germany) and then the FOR reagents were added to the template in the PCR reaction tube.

f. PCR Cycler

PCR's were carried out in PCR cyclers type Mastercycler or Mastercycler nexus gradient from Eppendorf AG (Hamburg, Germany).

7. Restriction Ezyme Digestion of DNA

The FastDigest restriction endonucleases (FD) and the associated buffer from ThermoFisher Scientific (Waltham, USA, Cat. No. FD0684) were used for restriction digestion of the plasmid DNA. The reactions were carried out according to the instructions of the manufacturer's manual.

8. Determining the Size of DNA Fragments

The size of DNA fragments was determined by automatic capillary electrophoresis using the QIAxcel from Qiagen (Hilden, Germany).

9. Purification of PCR Amplificates and Restriction Fragments

PCR amplificates and restriction fragments were cleaned up using the QIAquick PCR Purification Kit from Qiagen (Hilden, Germany; Cat. No. 28106), according to the manufacturer's instructions.

10. Determining DNA Concentration

DNA concentration was measured using the NanoDrop Spectrophotometer ND-1000 from PEQLAB Biotechnologie GmbH, since 2015 VWR brand (Erlangen, Germany).

11. Gibson Assembly

Expression vectors and vectors allowing integration of the desired deletion mutation into the chromosome were made using the method of Gibson et al. (Science 319, 1215-20, 2008). The Gibson Assembly Kit from New England BioLabs Inc. (Ipswich, USA; Cat. No. E2611) was used for this purpose. The reaction mix, containing the restricted vector and at least one DNA insert, was incubated at 50° C. for 60 min. 0.5 μl of the Assembly mixture was used for a transformation experiment.

12. Chemical Transformation of E. coli

a. Chemically competent E. coli Stellar™ cells were purchased from Clontech Laboratories Inc. (Mountain View, USA; Cat. No. 636763) and transformed according to the manufacturer's protocol (PT5055-2).

These cells were used as transformation hosts for reaction mixtures obtained by Gibson Assembly. The transformation batches were cultivated overnight for approximately 18 h at 37° C. and the transformants containing plasmids selected on LB agar supplemented with 50 mg/li kanamycin.

b. E. coli K-12 strain S17-1 was used as donor for conjugational transfer of plasmids based on pK18mobsacB from E. coli to C. glutamicum. Strain S17-1 is described by Simon, R. et al. (Bio/Technology 1, 784-794, 1983). It is available from the American Type Culture Collection under the access number ATCC47055.

Chemically competent E. coli S17-1 cells were made as follows: A preculture of 10 ml LB medium (10 ml liquid medium per 100 ml Erlenmeyer flask with 3 baffles) was inoculated with 100 μl bacterial suspension of strain S17-1 and the culture was incubated overnight for about 18 h at 37° C. and 250 rpm. The main culture (70 ml LB contained in a 250 ml Erlenmeyer flask with 3 baffles) was inoculated with 300 μl of the preculture and incubated up to an OD600 of 0.5-0.8 at 37° C. The culture was centrifuged for 6 min. at 4° C. and 4000 rpm and the supernatant was discarded. The cell pellet was resuspended in 20 ml sterile, ice-cold 50 mM CaCl₂ solution and incubated on ice for 30 min. After another centrifugation step, the pellet was resuspended in 5 ml ice-cold 50 mM CaCl₂ solution and the suspension incubated on ice for 30 min. The cell suspension was then adjusted to a final concentration of 20% glycerol (v/v) with 85% sterile ice-cold glycerol. The suspension was divided into 50 μl aliquots and stored at −80° C.

To transform S17-1 cells, the protocol according to Tang et al. (Nucleic Acids Res. 22(14), 2857-2858, 1994) with a heat shock of 45 sec. was used.

13. Conjugation of C. glutamicum

The pK18mobsacB plasmid system described by Schäfer et al, (Gene 145, 69-73, 1994) was used to integrate desired DNA fragments into the chromosome of C. glutamicum. A modified conjugation method of Schäfer et al. (Journal of Bacteriology 172, 1663-1666, 1990) was used to transfer the respective plasmid into the desired C. glutamicum recipient strain.

Liquid cultures of the C. glutamicum strains were carried out in BHI medium at 33° C. The heat shock was carried out at 48.5° C. for 9 min. Transconjugants resulting from a first recombination event were selected by plating the conjugation batch on EM8 agar (Table 12), which was supplemented with 25 mg/l kanamycin and 50 mg/l nalidixic acid. The EM8 agar plates were incubated for 72 h at 33° C.

TABLE 12
Composition of the EM8 agar.

| Components | Concentration (g/l) |
|---|---|
| Glucose (sterile-filtered) | 23 |
| CSL (corn steep liquor) | 30 |
| Peptone from soymeal (Merck, Germany) | 40 |
| (NH₄)₂SO₄ | 8 |
| Urea | 3 |
| KH₂PO₄ | 4 |
| MgSO₄•7 H₂O | 0.5 |
| FeSO₄•7 H₂O | 0.01 |
| CuSO₄•5 H₂O | 0.001 |
| ZnSO₄•7 H₂O | 0.01 |
| Calcium pantothenate, D(+) | 0.01 |
| Thiamine | 0.001 |
| Inositol | 0.1 |
| Nicotinic acid | 0.001 |
| Biotin (sterile-filtered) | 0.005 |
| CaCO₃ (autoclaved separately) | 1.6 |
| Agar-Agar (Merck, Germany) | 14 |

Sterile toothpicks were used to transfer the transconjugants onto BHI agar, which was supplemented with 25 mg/l kanamycin and 50 mg/l nalidixic acid. The agar plates were incubated for 20 h at 33° C.

The cultures of the respective transconjugants produced in this manner were then propagated further for 24 h at 33° C. in 10 ml BHI medium contained in 100 ml Erlenmeyer flasks with 3 baffles. To isolate clones having encountered a second recombination event an aliquot was taken from the liquid culture, suitably diluted and plated (typically 100 to 200 μl) on BHI agar which was supplemented with 10% saccharose. The agar plates were incubated for 48 h at 33° C. The colonies growing on the saccharose containing agar plates were then examined for the phenotype kanamycin sensitivity. To do so a toothpick was used to remove cell material from the colony and to transfer it onto BHI agar containing 25 mg/l kanamycin and onto BHI agar containing 10% saccharose. The agar plates were incubated for 60 h at 33° C. Transconjugant clones that proved to be sensitive to kanamycin and resistant to saccharose were examined for integration of the desired genetic feature into the chromosome by means of PCR.

14. Determining Nucleotide Sequences

Nucleotide sequences of DNA molecules were determined by Eurofins Genomics GmbH (Ebersberg, Germany) by cycle sequencing, using the dideoxy chain termination method of Sanger et al. (Proceedings of the National Academy of Sciences USA 74, 5463-5467, 1977), on Applied Biosystems® (Carlsbad, Calif., USA) 3730xl DNA Analyzers. Clonemanager Professional 9 software from Scientific & Educational Software (Denver, USA) was used to visualise and evaluate the sequences.

15. Transformation of C. glutamicum by Electroporation

Plasmid vectors based on pVWEx1 were transferred into cells of C. glutamicum using a modified electroporation method by Van der Rest et al. (Appl Microbiol Biotechnol 52, 541-545, 1999). To produce competent C. glutamicum cells the strains were propagated in BHIS medium (37 g/l BHI, 91 g/l sorbitol (Sigma Aldrich, St. Louis, USA)) by a preculture and a subsequent main culture. The preculture consisted of 10 ml BHIS medium contained in a 100 ml Erlenmeyer flask with 3 baffles. It was inoculated with 100 μl of a glycerol stock culture and incubated overnight for about 18 h at 33° C. and 200 rpm. The main culture consisted of 250 ml BHIS medium contained in a 1 l Erlenmeyer flask with 4 baffles. It was inoculated with 5 ml of the preculture and incubated for 4 h at 33° C. and 150 rpm to an OD600 of approx. 1.8.

The following working steps were carried out on ice using sterile, ice cold buffers or solutions resp. The main culture was centrifuged for 20 min. at 4° C. and 4000 rpm. The supernatant was discarded, the cell pellet resuspended in 2 ml TG buffer (1 mM Tris(hydroxymethyl)-aminomethane, 10% glycerol, adjusted to pH 7.5 with HCl) and another 20 ml TG buffer added to the cell suspension. This washing step was repeated twice. Said washing steps were followed by two further washing steps in which the TG buffer was replaced by a 10% (v/v) glycerol solution. After the final centrifugation step 2 ml 10% (v/v) glycerol were added to the cell pellet. The cell suspension obtained was then aliquoted in 100 μl portions and stored at −80° C.

The electroporation of the C. glutamicum strains was carried out as described by Van der Rest et al. Deviating from this procedure the cultivation temperature was 33° C. and the medium for agar plate cultures was BHI agar. Transformants were selected on BHI agar plates supplemented with 25 mg/l kanamycin.

16. Glycerol Stocks of E. coli and C. glutamicum Strains

For long time storage of E. coli - and C. glutamicum strains glycerol stocks were prepared. Selected E. coli clones were cultivated in 10 ml LB medium supplemented with 2 g/l glucose. Selected C. glutamicum clones were cultivated in two-fold concentrated BHI medium supplemented with 2 g/l glucose. Cultures of plasmid containing E. coli strains were supplemented with 50 mg/l kanamycin. Cultures of plasmid containing C. glutamicum strains were supplemented with 25 mg/l kanamycin. The medium was contained in 100 ml Erlenmeyer flasks with 3 baffles. It was inoculated with a loop of cells taken from a colony. The culture was then incubated for about 18 h at 37° C. and 200 rpm in the case of E. coli and 33° C. and 200 rpm in the case of C. glutamicum. After said incubation period 1.2 ml 85% (v/v) sterile glycerol were added to the culture. The obtained glycerol containing cell suspension was then aliquoted in 2 ml portions and stored at −80° C.

17. Cultivation System According to Wouter Duetz

The millilitre-scale cultivation system according to W. A. Duetz (Trends Microbiol. 2007; 15(10):469-75) was used to investigate the performance of the C. glutamicum strains constructed. For this purpose 24-deepwell microplates (24 well WDS plates) from EnzyScreen BV (Heemstede, Netherlands; Cat. no. CR1424) filled with 2.5 ml medium per deepwell were used.

Precultures of the strains were done in 10 ml two-fold concentrated BHI medium. The medium was contained in a 100 ml Erlenmeyer flask with 3 baffles. It was inoculated with 100 μl of a glycerol stock culture and the culture incubated for 24 h at 33° C. and 200 rpm.

After said incubation period the optical densities OD600 of the precultures were determined.

The main cultures were done by inoculating the 2.5 ml medium containing wells of the 24 Well WDS-Plate with an aliquot of the preculture to give an optical density OD600 of 0.1.

As medium for the main culture CGXII medium described by Keilhauer et al. (J. Bacteriol. 1993 September; 175(17): 5595-5603) was used. For convenience the composition of the CGXII medium is shown in table 13.

TABLE 13
Composition of Keilhauer's CGXII medium.

| Components | Concentration (g/l) |
|---|---|
| MOPS (3-(N-Morpholino)propanesulfonic acid) | 42 |
| (NH₄)₂SO₄ | 20 |
| Urea | 5 |
| KH₂PO₄ | 1 |
| K₂HPO₄ | 1 |
| MgSO₄•7 H₂O | 0.25 |
| CaCl₂ | 0.01 |
| FeSO₄•7 H₂O | 0.01 |
| MnSO₄ H₂O | 0.01 |
| ZnSO₄•7 H₂O | 0.001 |
| CuSO₄•5 H₂O | 0.0002 |
| NiCl₂ 6 H₂O | 0.00002 |
| Biotin (sterile-filtered) | 0.0002 |
| Protocatechuic acid (sterile-filtered) | 0.03 |
| Carbon source (sterile-filtered) | 20 |
| adjust the pH to 7 with NaOH | |

These main cultures were incubated for approximately 45 h at 33° C. and 300 rpm in an Infors HT Multitron standard incubator shaker from Infors GmbH (Bottmingen, Switzerland) until complete consumption of glucose.

The glucose concentration in the suspension was analysed with the blood glucose-meter OneTouch Vita® from LifeScan (Johnson & Johnson Medical GmbH, Neuss, Germany).

After cultivation the culture suspensions were transferred to a deep well microplate. A part of the culture suspension was suitably diluted to measure the OD600. Another part of the culture was centrifuged and the concentration of L-amino acids, e.g. L-lysine or L-valine, and residual glucose was analysed in the supernatant.

18. Amino Acid Analyser

The concentration of L-amino acids, in particular L-lysine, in the culture supernatants was determined by ion exchange chromatography using a SYKAM S433 amino acid analyser from SYKAM Vertriebs GmbH (Fürstenfeldbruck, Germany). As solid phase a column with spherical, polystyrene-based cation exchanger (Peek LCA N04/Na, dimension 150×4.6 mm) from SYKAM was used. Depending on the L-amino acid the separation took place in an isocratic run using a mixture of buffers A and B for elution or by gradient elution using said buffers. As buffer A an aquous solution containing in 20 l 263 g trisodium citrate, 120 g citric acid, 1100 ml methanol, 100 ml 37% HCl and 2 ml octanoic acid (final pH 3.5) was used. As buffer B an aquous solution containing in 20 l 392 g trisodium citrate, 100 g boric acid and 2 ml octanoic acid (final pH 10.2) was used. The free amino acids were coloured with ninhydrin through post-column derivatization and detected photometrically at 570 nm.

19. Glucose Determination with Continuous Flow System (CFS)

A SANplus multi-channel continuous flow analyser from SKALAR analytic GmbH (Erkelenz, Germany) was used to determine the concentration of glucose in the supernatant. Glucose was detected with a coupled-enzyme assay (Hexokinase/Glucose-6-Phosphate-Dehydrogenase) via NADH formation.

20. Minimal Inhibitory Concentration Test (MIC-Test)

The minimal inhibitory concentrations (MIC's) of lincomycin for different strains were determined in liquid culture using a serial dilution assay with dilution steps of 1:2 covering a concentration range of 250 μg/ml, 125 μg/ml etc. down to 0.12 μg/ml.

For this purpose a first well of a microliter plate was filled with 360 μl of BHI medium supplemented with 0.3 mM IPTG and 250 μg/ml lincomycin-hydrochloride. A volume of 180 μl was removed and placed into a second well previously filled with 180 μl BHI medium supplemented with 0.3 mM IPTG. Thus a second well containing BHI medium supplemented with 0.3 mM IPTG and 125 μg/ml lincomycin-hydrochloride was prepared. From said second well 180 ∞l were removed and placed into a third well previously filled with 180 μl BHI medium supplemented with 0.3 mM IPTG. These dilution steps were repeated several times to cover the concentration range mentioned above. Thus wells containing 180 μl of the respective test medium were obtained. A control medium without lincomycin-hydrochloride was included in the test procedure.

A preculture of the strain to be tested was made in BHI medium (as described under cultivation) supplemented with 250 μg/ml kanamycin in case of plasmid containing strains). The preculture was centrifuged and the cell pellet resuspended in 0.9% (w/v) NaCl solution to give an OD660 of 1.0. A volume of 20 μl of the suspension was used to inoculate the test medium contained in the wells. The micro titer plate was incubated at 300 rpm and 33° C. in an Infors HT Multitron standard incubator shaker from Infors GmbH (Bottmingen, Switzerland). After 20 h the optical densities were measured. The test medium having the lowest concentration of lincomycin-hydrochloride preventing growth of the culture is given as MIC.

### B) Experimental Results

### Example 1

### Sequence of the lmrB Gene of Strains DM1933, DM1797 and DM1547

Strain DM1933 is an L-lysine producer described by Blombach et al. (Applied and Environmental Microbiology 75(2), 419-427, 2009). It is deposited according to the Budapest treaty at the DSMZ under accession number DSM25442.

The nucleotide sequence of the chromosome of strain DM1933 as well as DM1797 and DM1547 was determined by Illumina whole-genome sequencing technology (Illumina Inc., San Diego, Calif., US). See e.g. Benjak et al. (2015) Whole-Genome Sequencing for Comparative Genomics and De Novo Genome Assembly. In: Parish T., Roberts D. (eds) Mycobacteria Protocols. Methods in Molecular Biology, Vol 1285. Humana Press, NY, US) and Bennet, S. (Pharmacogenomics 5(4), 433-438, 2004).

It was found that the nucleotide sequence of the lmrB coding sequence of DM1933 including the nucleotide sequence upstream and downstream thereof is identical to that of ATCC13032 shown in SEQ ID NO:7.

Strain DM1797 is an L-lysine producer described in U.S. Pat. No. 7,338,790 B2 (see column 30). It is deposited according to the Budapest treaty at the DSMZ under accession number DSM16833 on 28 Oct. 2004. DM1797 is an aminoethylcystein resistant mutant of strain ATCC13032 obtained after N′-methyl-N-nitro-nitrosoguanidine mutagenesis. DM1797 differs from ATCC13032 only in that the aspartokinase gene in the chromosome of DM1797 is a variant coding for a feedback resistant aspartokinase polypeptide. Said feedback resistant aspartokinase polypeptide has the amino acid sequence of SEQ ID NO:15 of the sequence listing, wherein the amino acid L-threonine (Thr) at position 311 of the amino acid sequence is replaced by L-isoleucine (Ile). In U.S. Pat. No. 7,338,790 the abbreviation “lysC T311l” is used to indicate said exchange.

The nucleotide sequence of the lmrB coding sequence of DM1797 including the nucleotide sequence upstream and downstream thereof is identical to that of ATCC13032 shown in SEQ ID NO:7.

Strain DM1547 is an L-lysine producer described in EP 1 239 040 A2. It is deposited according to the Budapest treaty at the DSMZ under accession number DSM13944. DM1547 is an aminoethylcystein resistant mutant of strain ATCC13032 obtained after several rounds of mutagenesis and screening.

The nucleotide sequence of the lmrB coding sequence of DM1547 including the nucleotide sequence upstream and downstream thereof is identical to that of ATCC13032 shown in SEQ ID NO:7 with the exception that the nucleobase at position 1311 is thymine.

Strain DM1933 also contains said variant of the aspartokinase gene. It is abbreviated as “lysC(T311l)” by Blombach et al. (see table 1 of Blombach et al. APPLIED AND ENVIRONMENTAL MICROBIOLOGY, January 2009, p. 419-427).

### Example 2

### Construction of Plasmid pK18mobsacB_DlmrB

Plasmid pK18mobsacB_Dcmr was constructed to enable incorporation of a deletion, comprising the lmrB coding sequence and the adjoining stop codon accompanied by the insertion of the recognition site for the restriction endonuclease EcoRV, into the chromosome of a desired C. glutamicum strain. The plasmid is based on the mobilizable vector pK18mobsacB described by Schäfer et al. (Gene 145, 69-73, 1994). For the construction of pK18mobsacB_Dcmr the Gibson assembly method was used.

For this purpose three polynucleotides or DNA molecules resp. were generated: One polynucleotide called lmrB_up comprising the upstream sequence (5′-flanking sequence) and a second polynucleotide called lmrB_down comprising the downstream sequence (3′-flanking sequence) of the coding sequence of lmrB. The third polynucleotide was plasmid pK18mobsacB linearized by treatment with restriction endonuclease Xbal. The polynucleotides ImrB_up and lmrB_down were fused during the Gibson assembly process to give the polynucleotide DlmrB, comprising the nucleotide sequence of SEQ ID NO:13, contained in pK18mobsacB_DlmrB.

Polynucleotides lmrB_up and lmrB_down were synthesized by PCR using total DNA isolated from a C. glutamicum ATCC13032 culture as template. For PCR the Phusion Kit was used with an elongation step (see table 9, step 4) of 15 sec. For amplification of the downstream sequence (polynucleotide lmrB_down) the primers 1f-DlmrB and 1r-DlmrB and for amplification of the upstream sequence (polynucleotide lmrB_up) the primers 2f-DlmrB and 2r-DlmrB were used (table 14). The primers are also shown in SEQ ID NO:17 to SEQ ID NO:20 of the sequence listing.

TABLE 14
List of primers used and size of amplificates during Phusion-
Kit PCP.

| Synthesis of | | | Size |
|---|---|---|---|
| amplificate | Name | Sequence | [bp] |
| lmrB_down | 1f-DlmrB | AGCTCGGTACCCGGGGATCCTAGATC | 838 |
| | | TCCATCCCCACGTTC | |
| | 1r-DlmrB | GCTTCATGCTGTGAAAGGCCGATATCA | |
| | | ATGGGGCAGAAGGTCATTG | |
| lmrB_up | 2f-DlmrB | CAATGACCTTCTGCCCCATTGATATCG | 834 |
| | | GCCTTTCACAGCATGAAGC | |
| | 2r-DlmrB | TGCATGCCTGCAGGTCGACTAAGGCT | |
| | | ACGTTCAAAATCC | |

The nucleotide sequence of the amplificate lmrB_up is shown in SEQ ID NO:21. The nucleotide sequence of the amplificate lmrB_down is shown in SEQ ID NO22.

Amplificate lmrB_up contains a sequence of 788 nucleotides of the upstream region of the lmrB coding sequence of ATCC13032. At its 5′-end it is equipped with a sequence overlapping with a sequence of pK18mobsacB cut with Xbal. At its 3′-end it is equipped with a sequence overlapping with a sequence of the amplificate lmrB_down. Said sequence at the 3′-end contains the recognition site for the restriction endonuclease EcoRV.

Amplificate lmrB_down contains a sequence of 791 nucleotides of the downstream region of the lmrB coding sequence of ATCC13032. At its 5′-end it is equipped with a sequence overlapping with a sequence of the amplificate lmrB_up. Said sequence at the 5′-end contains the recognition site for the restriction endonuclease EcoRV. At its 3′-end it is equipped with a sequence overlapping with a sequence of pK18mobsacB cut with Xbal. Said overlapping sequences are required for the Gibson assembly technique.

Plasmid pK18mobsacB was linearized with the restriction endonuclease Xbal. The digestion mixture was controlled by capillary electrophoresis, purified and the DNA concentration quantified. To assemble the plasmid pK18mobsacB_DlmrB the three polynucleotides i.e. the vector pK18mobsacB cut with Xbal, the amplificate lmrB_up and the amplificate lmrB_down were mixed using the Gibson Assembly Kit. The assembly mixture thus obtained was used to transform chemically competent E. coli Stellar™ cells.

Fifty kanamycin resistant transformants were analyzed by colony PCP using the Taq Kit and the primers pCV22_1.p and pCV22_2.p according to the protocol shown in table 10. The primers are shown in table 15 and under SEQ ID NO:23 and SEQ ID NO:24 of the sequence listing. The size of the amplificates was controlled by capillary electrophoresis.

TABLE 15
List of primers used for colony PCR and size of amplificate
during Tag Kit PCR.

| indication for | | | size |
|---|---|---|---|
| the presence of | name | sequence | [bp] |
| DlmrB | pCV22_1.p | AGGTTTCCCGACTGGAAAGC | 1872 |
| | pCV22_2.p | TGCAAGGCGATTAAGTTGGG | |

One of the transformants thus characterized containing a plasmid of the desired size was called Stellar/pK18mobsacB_DlmrB and saved as a glycerol stock.

DNA of the plasmid pK18mobsacB_DlmrB was isolated from said transformant and the polynucleotide DlmrB created within pK18mobsacB during the Gibson assembly was analyzed by Sanger sequencing using the primers pVW_1.p and M13For shown in table 16. Said primers are also shown under SEQ ID NO:25 and SEQ ID NO:26 of the sequence listing.

TABLE 16
List of primers used for Sanger sequencing.

| detection of | name | sequence |
|---|---|---|
| DlmrB | pVW_1.p | GTGAGCGGATAACAATTTCACAC |
| | M13For | GTAAAACGACGGCCAG |

The analysis of the nucleotide sequence thus obtained showed that the polynucleotide DlmrB contained in pK18mobsacB_DlmrB had the nucleotide sequence presented in SEQ ID NO:13.

### Example 3

### Construction of Strain DM1933_ΔlmrB::EcoRV

The plasmid pK18mobsacB_DlmrB was used to incorporate the deletion comprising the complete lmrB coding sequence and the adjoining stop codon accompanied by the insertion of the recognition site for the restriction enzyme EcoRV into the chromosome of the L-lysine producer DM1933.

Said deletion comprising the complete lmrB coding sequence and the adjoining stop codon accompanied by the insertion of the recognition site for the restriction enzyme EcoRV is abbreviated as ΔlmrB::EcoRV or deltalmrB::EcoRV when appropriate.

Chemically competent cells of E. coli strain S17-1 were transformed with the plasmid DNA obtained in example 2. The modified conjugation method from Schäfer et al. (Journal of Bacteriology 172, 1663-1666, 1990) as described in materials and methods was used for conjugal transfer into the strain DM1933 and for selection for transconjugant clones by virtue of their saccharose resistance and kanamycin sensitivity phenotype.

Transconjugant clones were analyzed by colony PCR using the primers NCgl2591_fw and NCgl2591_rev listed in table 17, followed by size determination of the amplificates by capillary electrophoresis. The primers are also shown in SEQ ID NO:27 and SEQ ID NO:28 of the sequence listing. For PCR the Sapphire Mix (see table 11) was used.

TABLE 17
List of primers used for colony PCR and size of amplificate
during Sapphire Mix PCR.

| amplification/ | | | size |
|---|---|---|---|
| detection of | name | sequence | [bp] |
| ΔlmrB::EcoRV | NCgl2591_fw | GGCGAGAACAGAACCTGGAG | 255 |
| | NCgl2591_rev | CTCAAGCAAGCCGCCTAAAC | |

One of the transconjugant clones thus characterized was called DM1933_ΔlmrB::EcoRV. A glycerol stock culture of the transconjugant clone was prepared and used as starting material for further investigations.

The nucleotide sequence of the chromosomal region of strain DM1933_ΔlmrB::EcoRV containing the mutated nucleotide sequence, i.e. lack (deletion) of the lmrB coding sequence and the adjoining stop codon accompanied by insertion of the recognition site for the restriction endonuclease EcoRV, was analyzed by Sanger sequencing.

For this purpose a PCR amplificate was produced spanning the site of mutation. A colony PCR was done using the primers NCgl2591_fwd1 and NCgl2593_rev1 (see table 18) and the Phusion Kit (see table 9) with an elongation time of 45 sec. (see step 4 of table 9). The amplificate obtained was then sequenced using the primers NCgl2591_fwd2 and NCgl2593_rev2 (see table 18). The nucleotide sequences of the primers used in this context are also shown in SEQ ID NO:29 to 32.

TABLE 18
List of primers used for colony PCR and Sanger sequencing.

| amplification/ | | | size |
|---|---|---|---|
| detection of | name | sequence | [bp] |
| ΔlmrB::EcoRV | NCgl2591_fwd1 | CAAGAGATCGTCCTCGATTC | 1532 |
| | NCgl2593_rev1 | GTCCGGGATGCGATATAAAG | |
| ΔlmrB::EcoRV | NCgl2591_fwd2 | TTCCAGCAGGTTGGAGATTG | |
| ΔlmrB::EcoRV | NCgl2593_rev2 | GGCGGCTGTGGTCACTATTG | |

The nucleotide sequence obtained is shown in SEQ ID NO:33. It contains the nucleotide sequences identified in tables 2, 3, 4, 5 and 6.

The result showed that strain DM1933_←lmrB::EcoRV contained the desired mutation, or the desired mutated nucleotide sequence resp., in its chromosome. Hence the lmrB gene of strain DM1933 was replaced by the ΔlmrB::EcoRV mutation.

### Example 4

### L-lysine Production by Strain DM1933_ΔlmrB::EcoRV

Strains DM1933_ΔlmrB::EcoRV and DM1933 (as reference) were analyzed for their ability to produce L-lysine from glucose by batch cultivation using the system of Wouter Duetz.

As medium CGXII containing 20 g/l glucose as carbon source was used. The cultures were incubated for 45 h until complete consumption of glucose as confirmed by glucose analysis using blood glucose-meter and the concentrations of L-lysine and optical density OD660 were determined. The result of the experiment is presented in table 19.

TABLE 19
L-lysine production by strain DM1933_ΔlmrB::EcoRV.

| strain | L-lysine¹ (g/l) | OD660 |
|---|---|---|
| DM1933 | 5.1 | 5.3 |
| DM1933_ΔlmrB::EcoRV | 5.9 | 4.0 |

¹as L-lysine × HCl

DM1933 contains in its chromosome a variant of the aspartokinase gene encoding a feedback resistant aspartokinase polypeptide. DM1933_ΔlmrB::EcoRV also contains in its chromosome a variant of the aspartokinase gene encoding a feedback resistant aspartokinase polypeptide and its lmrB gene was replaced by the ΔlmrB::EcoRV mutation.

The experiment showed that L-lysine formation was higher in strain DM1933_ΔlmrB::EcoRV as compared to the reference strain DM1933.

### Example 5

### Construction of Strains ATCC13032_ΔlmrB::EcoRV and DM1797_ΔlmrB::EcoRV

The strains were constructed and analyzed as described in example 3.

### Example 6

### L-Lysine production by Strains ATCC13032_ΔlmrB::EcoRV and DM1797_ΔlmrB::EcoRV

The production test was done as described in example 4. The result is shown in table 20.

TABLE 20
L-lysine production by strains ATCC13032_ΔlmrB::EcoRV
and DM1797_ΔlmrB::EcoRV

| strain | L-lysine¹ (g/l) | OD660 |
|---|---|---|
| ATCC13032 | nd² | 3.4 |
| ATCC13032_ΔlmrB::EcoRV | nd² | 3.3 |
| DM1797 (=“ATCC13032_lysC T311I”) | 3.4 | 4.2 |
| DM1797_ΔlmrB::EcoRV | 3.8 | 3.9 |

¹as L-lysine × HCl
²not detectable

Strain ATCC13032 (also available as DSM20300) is the taxonomic (wild-)type strain of the species Corynebacterium glutamicum.

Strain ATCC13032_ΔlmrB::EcoRV differs from ATCC13032 only in that its lmrB gene was replaced by the ΔlmrB::EcoRV mutation.

DM1797 differs from ATCC13032 only in that the aspartokinase gene (lysC) in the chromosome of DM1797 is a variant coding for a feedback resistant aspartokinase polypeptide (“lysC T311l”).

DM1797_ΔlmrB::EcoRV also contains in its chromosome a variant of the aspartokinase gene encoding a feedback resistant aspartokinase polypeptide (“lysC T311l”) and its lmrB gene was replaced by the ΔlmrB::EcoRV mutation.

The experiment showed that L-lysine formation was higher in strain DM1797_ΔlmrB::EcoRV as compared to the reference strain DM1797.

### Example 7

### Construction of Plasmid pVWEx1_lmrB^{ATCC14067}

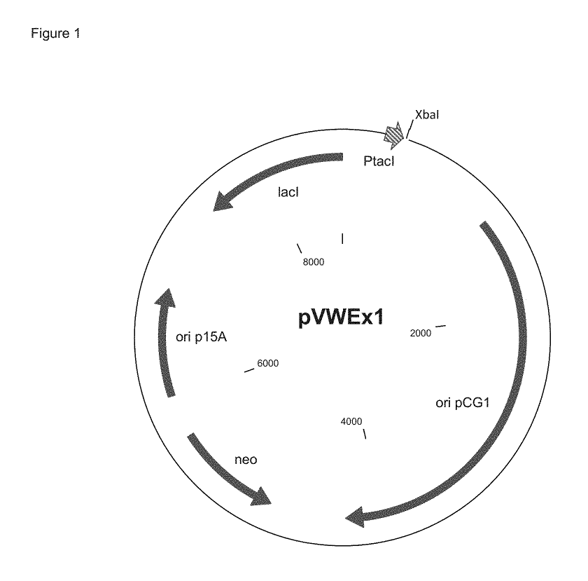

A polynucleotide called lmrB^{ATCC14067} encoding the LmrB polypeptide of strain ATCC14067 (LmrB^{ATCC14067}) shown in SEQ ID No:6 and SEQ ID NO:12 was cloned into the shuttle vector pVWEx1 described by Peters-Wendisch et al. (Journal of Molecular Microbiology and Biotechnology 3, 295-300, 2001). The nucleotide sequence of pVWEx1 is available at the GenBank database under accession number MF034723. A map of plasmid pVWEx1 is shown in FIG. 1.

The DNA molecule lmrB^{ATCC14067} was produced by PCR using total DNA of strain ATCC14067 as template and the primers 1f-lmrB and 1r-lmrB presented in table 21. The two primers are also shown under SEQ ID NO:34 and SEQ ID NO:35 of the sequence listing. For amplification the Phusion Kit (table 9) was used with an elongation step of 45 sec. (see step 4 of table 9).

TABLE 21
List of primers used for Phusion Kit PCR.

| amplifi- | | | size |
|---|---|---|---|
| cation of | name | sequence | [bp] |
| lmrB^{ATCC14067} | 1f-lmrB | GCCTGCAGGTCGACTATGGATTC | 1477 |
| | | CCAAATTAATACTC | |
| | 1r-lmrB | GGTACCCGGGGATCCTTTAGTGA | |
| | | GTTTCTTCCTCG | |

The size of the amplificate was analyzed by capillary electrophoresis. Plasmid pVWEx1 was treated with restriction endonuclease Xbal. The digestion mixture was controlled by capillary electrophoresis, purified and the DNA concentration quantified.

To assemble plasmid pVWEx1_lmrB^{ATCC14067} the two DNA molecules, i.e. lmrB^{ATCC14067} and pVWEx1 cut with Xbal, were mixed using the Gibson Assembly Kit. The assembly mixture thus obtained was used to transform chemically competent E. coli Stellar™ cells.

Transformants were analyzed by colony PCR using the Taq Kit (see table 10) and the primers pVW_1.p and pVW_2.p presented in table 22. The primers are also shown in SEQ ID NO:25 and SEQ ID NO:36.

TABLE 22
List of primers used for colony PCR and Sanger sequencing.

| detection of | name | sequence | size [bp] |
|---|---|---|---|
| lmrB^{ATCC14067} | pVW_1.p | see table 8 | 1578 |
| | pVW_2.p | CGACGGCCAGTGAATTCGAG | |

One of the transformants thus characterized, containing the plasmid pVWEx1_lmrB^{ATCC14067} was called Stellar/pVWEx1_lmrB^{ATCC14067} and saved as a glycerol stock.

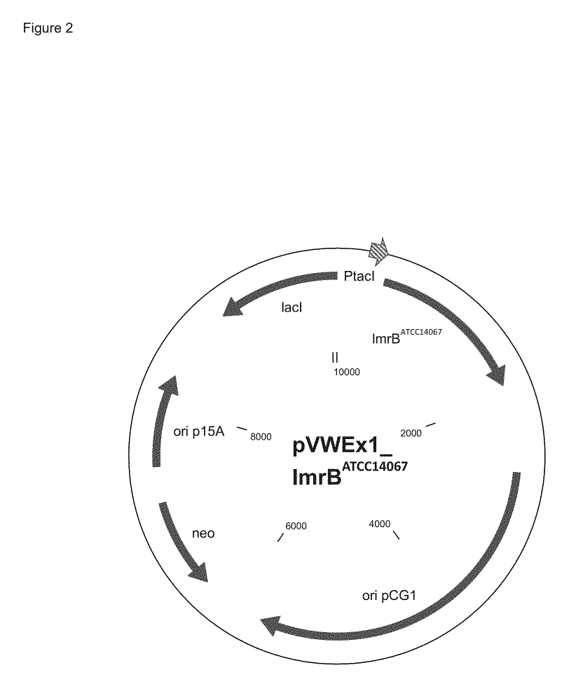

DNA of the obtained plasmid pVWEx1_lmrB^{ATCC14067} was isolated and used for further investigation. A map of pVWEx1_lmrB^{ATCC14067} is shown in FIG. 2.

The nucleotide sequence of lmrB^{ATCC14067} being inserted within pVWEx1 was analyzed by Sanger sequencing using the primers pVW_1.p and pVW_2.p (table 22) and found to contain the desired nucleotide sequence.

Example 8

### Construction of Strains ATCC13032/pVWEx1; ATCC13032/pVWEx1_lmrB^{ATCC14067}; DM1933_ΔlmrB::EcoRV/pVWEx1 and DM1933_ΔlmrB::EcoRV/pVWEx1_lmrB^{ATCC14067}

As host for assessing the ability of the constructed plasmid pVWEx1_lmrB^{ATCC14067} to confer lincomycine resistance to the species C. glutamicum, strains ATCC13032 and DM1933_ΔlmrB::EcoRV were chosen.

Strains ATCC13032 and DM1933_ΔlmrB::EcoRV (see example 3) were transformed with isolated plasmid DNA of pVWEx1 and pVWEx1_ΔlmrB^{ATCC14067} by electroporation. Selection for transformants, propagation of the transformants and preparation of glycerol stock cultures was done as described under materials and methods and in the presence of kanamycin.

The transformants were analyzed by colony PCR (Sapphire Mix) using the primers pVW_1.p and pVW_2.p (table 22) for the presence (pVWEx1_lmrB^{ATCC14067}) or absence (pVWEx1) of the desired nucleotide sequence.

Thus strains ATCC13032/pVWEx1; ATCC13032/pVWEx1_lmrB^{ATCC14067}; DM11933_ΔlmrB::EcoRV/pVWEx1 and DM1933_ΔlmrB::EcoRV/pVWEx1_lmrB^{ATCC14067} were obtained.

### Example 9

### MIC of Lincomycin for Different C. glutamicum Strains Overexpressing the lmrB Gene of Strain ATCC14067

The MIC's of lincomycin for strains ATCC13032/pVWEx1; ATCC13032/pVWEx1_lmrB^{ATCC14067}; DM1933_ΔlmrB::EcoRV/pVWEx1 and DM1933_ΔlmrB::EcoRV/pVWEx1_lmrB^{ATCC14067} constructed in example 8 were determined as described in materials and methods.

The result of the experiment is presented in table 23. It shows that the lmrB gene of strain ATCC14067 (lmrB^{ATCC14067}) encoding the polypeptide shown in SEQ ID NO:6 and SEQ ID NO:12 confers increased resistance to lincomycine to the C. glutamicum host.

TABLE 23
Determination of the MIC of
lincomycin¹ for different C. glutamicum strains.

| strain | MIC (μg/ml) |
|---|---|
| ATCC13032/pVWEx1 | 15.6 |
| ATCC13032/pVWEx1_lmrB^{ATCC14067} | 125 |
| DM1933_ΔlmrB::EcoRV/pVWEx1 | 7.8 |
| DM1933_ΔlmrB::EcoRV/pVWEx1_lmrB^{ATCC14067} | 62.5 |

¹as lincomycin-hydrochloride

This result showed that the LmrB polypeptide of strain ATCC14067 (LmrB^{ATCC14067}) has the activity of conferring lincomycin resistance to C. glutamicum.

### Example 10

### Construction of Plasmid pVWEx1_lmrB^{DM1547}

A polynucleotide called lmrB^{DM1547} encoding the LmrB polypeptide of strain DM1547 was cloned into the shuttle vector pVWEx1.

The DNA molecule lmrB^{DM1547} was produced by PCR using total DNA of strain DM1547 as template and the primers 1f-lmrB and pVT_rev presented in table 24. The two primers are also shown under SEQ ID NO:34 and SEQ ID NO:37 of the sequence listing. For amplification the Phusion Kit (table 9) was used with an elongation step of 45 sec. (see step 4 of table 9).

TABLE 24
List of primers used for Phusion Kit PCR.

| amplifi- | | | size |
|---|---|---|---|
| cation of | 1ame | sequence | [bp] |
| lmrB^{DM1547} | 1f-lmrB | see table 21 | 1477 |
| | pVT_rev | GGTACCCGGGGATCCTTTAGTGAGCTTCTTCCTC | |
| | | G | |

All other technical procedures including PCR amplification and pVWEx1 treatment with Xbal, cloning procedure and analysis of transformants were carried out essentially as described in example 7. One of the transformants containing the plasmid pVWEx1_lmrB^{DM1547} was called Stellar/pVWEx1_lmrB^{DM1547} and saved as a glycerol stock.

DNA of the obtained plasmid pVWEx1_lmrB^{DM1547} was isolated and used for further investigation. The nucleotide sequence of lmrB^{DM1547} being inserted within pVWEx1 was analyzed by Sanger sequencing using the primers pVW_1.p and pVW_2.p (see table 22) and found to contain the desired nucleotide sequence.

### Example 11

### Construction of Strain DM1933_ΔlmrB::EcoRV/pVWEx1_lmrB^{DM1547}

As host for assessing the ability of the constructed plasmid pVWEx1_lmrB^{DM1547} to confer lincomycine resistance to the species C. glutamicum, strain DM1933_ΔlmrB::EcoRV was chosen.

The technical procedures including transformant characterization were essentially the same as described in example 8.

As result of this experiment strain DM1933_ΔlmrB::EcoRV/pVWEx1_lmrB^{DM1547} was obtained.

### Example 12

### MIC of Lincomycin for Strain DM1933_ΔlmrB::EcoRV/pVWEx1_lmrB^{DM1547} Overexpressing the lmrB Gene of Strain DM1547

The MIC's of lincomycin for strains DM1933_ΔlmrB::EcoRV/pVWEx1 and DM1933_ΔlmrB::EcoRV/pVWEx1_lmrB^{DM1547} constructed in examples 8 and 11 were determined as described in materials and methods.

The result of the experiment is presented in table 25. It shows that the lmrB gene of strain DM1547 (lmrB^{DM1547}) encoding the polypeptide shown in SEQ ID NO:8, wherein valine is contained at position 104, confers increased resistance to lincomycine to the C. glutamicum host.

TABLE 25
Determination of the MIC of
lincomycin¹ for different C. glutamicum strains.

| strain | MIC (μg/ml) |
|---|---|
| DM1933_DlmrB/pVWEx1 | 7.8 |
| DM1933_DlmrB/pVWEx1_lmrB^{DM1547} | 62.5 |

¹as lincomycin-hydrochloride

This result showed that the LmrB polypeptide of strain DM1547 (LmrB^{DM1547}) has the activity of conferring lincomycin resistance to C. glutamicum.

Numerous modifications and variations on the present invention are possible in light of the above teachings. It is therefore to be understood that within the scope of the appended claims, the invention may be practiced otherwise than as specifically described herein.

### LIST OF ABBREVIATION IN THE FIGURES

lacl gene coding for the Lacl repressor

lmrB^{ATCC14067} gene coding for the LmrB polypeptide of strain ATCC14067

MCS multiple cloning site

neo gene coding for aminoglycoside 3′-phosphotransferase

ori p15A origin of replication of E. coli plasmid p15A

ori pCG1 origin of replication of C. glutamicum plasmid pCG1

Ptacl sequence of promoter Ptacl (De Boer et al., Proceedings of the National Academy of Sciences USA 80, 21-25, 1983)

Xbal sequence recognized by the restriction endonuclease Xbal

1. A method for the fermentative production of an L-amino acid, the L-amino acid selected from the group consisting of L-lysine, L-threonine and L-isoleucine, the method comprising:
a) cultivating a bacterium of the genus Corynebacterium having the ability to excrete said L-amino acid in a suitable medium under suitable conditions,
b) accumulating said L-amino acid in the medium to form an L-amino acid-containing fermentation broth,
wherein in said bacterium a polynucleotide coding for a polypeptide is modified by deleting or replacing at least parts of said polynucleotide,
wherein said polypeptide is at least 90% identical to the amino acid sequence of SEQ ID NO:8 and confers a resistance to at least one lincosamide selected from the group consisting of lincomycin and clindamycin, and
wherein said bacterium comprises at least one copy of a gene coding for a feedback-resistant aspartokinase.

2. The method as claimed in claim 1, wherein said polynucleotide is modified by deleting at least one or two nucleotides in the part of the coding sequence of said polynucleotide corresponding to amino acids of positions 1 to 258 of the amino acid sequence according to SEQ ID NO:8.

3. The method as claimed in claim 1, wherein said polynucleotide is modified by deleting at least one or two nucleotides in the part of the coding sequence of said polynucleotide corresponding to amino acids of positions 210 to 258 of the amino acid sequence according to SEQ ID NO:8.

4. The method as claimed in claim 1, wherein said polynucleotide is modified by deleting at least its complete coding sequence.

5. The method as claimed in claim 4, wherein said polynucleotide is modified by deleting at least its complete coding sequence and the adjoining stop codon.

6. The method as claimed in claim 1, wherein the modification of said polynucleotide results in an insertion of a recognition site for the restriction enzyme EcoRV in said polynucleotide.

7. The method as claimed in claim 1, wherein said polynucleotide is modified by inserting a gene coding for a polypeptide of the biosynthetic pathway of an L-amino acid into the part of the coding sequence corresponding to amino acids of positions 175 to 258 of the amino acid sequence according to SEQ ID NO:8.

8. The method as claimed in claim 1, wherein said polynucleotide is modified by substituting one or more codons coding for an amino acid of said polypeptide with or by inserting a TAA-, TGA- or TAG-stop codon in the part of coding sequence corresponding to amino acids of positions 1 to 258 of the amino acid sequence according to SEQ ID NO: 8.

9. The method as claimed in claim 1, wherein said L-amino acid is L-lysine.

10. The method as claimed in claim 1, wherein the gene coding for the feedback-resistant aspartokinase begins with an atg start codon.

11. The method as claimed in claim 1, wherein the amino acid sequence of said feedback resistant aspartokinase polypeptide comprises the amino acid sequence of SEQ ID NO:16 containing isoleucine at position 311 instead of threonine.

12. The method as claimed in claim 1, wherein said polypeptide confers a resistance to lincomycin.

13. The method as claimed in claim 1, wherein the bacterium is a bacterium of the species Corynebacterium glutamicum.

14. The method as claimed in claim 1, wherein said polypeptide, which is at least 90% identical to the amino acid sequence of SEQ ID NO:8, has an amino acid sequence elected from the group consisting of SEQ ID NO:8, SEQ ID NO:8 wherein valine is contained at position 104, SEQ ID NO:10, and SEQ ID NO:12.

15. The method as claimed in claim 1, wherein the bacterium excretes at least 0.1 g/l of the L-amino acid.


SEQUENCE LISTING (37 sequences)

SEQ ID NO: 1 - protein, 481 aa, Corynebacterium glutamicum
  misc_feature (1)..(481): amino acid sequence disclosed in GenBank accession number AF237667
  misc_feature (1)..(481): Strain of Corynebacterium glutamicum used in Kim et al, referred to as ASO19E12
MDSQINTQTSPAAAKLPREVVVVLSILVVSAMIMILNETILSVALPSIMEDFSVPETTAQWLTTGFMLTMAVVIPTTGYLLDRFSTKTIFVTALLFFTVGTLTAALAPTFAVLLGARIVQAVGTALVMPLLMTVTLTVVPAERRGSMMGIISIVISVAPALGPTLSGVILNSLTWHWLFWMMLPIVVIALVIGFFLIKNIGETKITPLDVLSVILSVFAFGGLVYGFSSFGAILEGEGTVGIFAIVVGAIALLIFALRQHQLGKQDKALMDLRAFKVRNFSFSLTTILLAFGAMLGTVMVLPIYLQTSLGVTALVTGLVVMPGGLLQGLISPFIGRFYDKVGPRPLLIPGAIALAIAASSMTFLNENSPVWMVVVMHVVFSIGMCLMMTPLMTTALGALPKHLYGHGSAILNTFQQLAGAAGTAIMIAALSFGTSIAASSGSAHAEAVAAGTKVAFIAGAIIAVIALVVSLFVTRVEEEAH

SEQ ID NO: 2 - protein, 481 aa, Corynebacterium glutamicum; identical to SEQ ID NO: 1
  MISC_FEATURE (1)..(481): amino acid sequence disclosed in GenBank accession number BAC00079
  MISC_FEATURE (1)..(481): the encoded amino acid sequence of a protein defined as “Permeases of the major facilitator superfamily” of Corynebacterium glutamicum of ATCC13032, disclosed in Ikeda and Nakagawa

SEQ ID NO: 3 - protein, 481 aa, Corynebacterium glutamicum
  misc_feature (1)..(481): amino acid sequence disclosed in GenBank accession number BAV24312
  misc_feature (1)..(481): the encoded amino acid sequence of a protein defined as “lincomycin resistance protein lmrB” of Corynebacterium glutamicum ssp. lactofermentum strain AJ1511, disclosed in Nishio et al.
MDSQINTQTSPAAAKLPREVVVVLSILVVSAMIMILNETILSVALPSIMEDFSVPETTAQWLTTGFMLTMAVVIPTTGYLLDRFSTKTIFVTALLFFTVGTLTAALAPTFAVLLGARIVQAVGTALVMPLLMTVTLTVVPAERRGSMMGIISIVISVAPALGPTLSGVILNSLTWHWLFWMMLPIVVIALVIGYFLIQNIGETKITPLDVLSVILSVFAFGGLVYGFSSFGAILEGEGTVGILAIVVGAIALLIFALRQHQLGKQDKALMDLRAFKVRNFSFSLTTILLAFGAMLGTVMVLPIYLQTSLGVTALVTGLVVMPGGLLQGLISPFIGRFYDKVGPRPLLIPGAIALAIAASSMTFLNENSPVWMVVVMHVVFSIGMCLMMTPLMTTALGALPKHLYGHGSAILNTFQQLAGAAGTAIMIAALSFGTSIAASSGSAHAEAVAAGTKVAFIAGAIIAVIALVVSLFVTRVEEETH

SEQ ID NO: 4 - protein, 481 aa, Corynebacterium glutamicum; identical to SEQ ID NO: 3
  misc_feature (1)..(481): amino acid sequence disclosed in GenBank accession number ANU34602
  misc_feature (1)..(481): the amino acid sequence of the encoded LmrB polypeptide from ATCC13869

SEQ ID NO: 5 - protein, 481 aa, Corynebacterium glutamicum
  misc_feature (1)..(481): amino acid sequence disclosed in GenBank accession number BAF55600
  misc_feature (1)..(481): the encoded amino acid sequence of a “hypothetical protein cgR_2586” from Corynebacterium glutamicum R having a region named “MFS”
MDSQINTQTSPATAKLPREVVVVLSILVISAMIMILNETILSVALPSIMADFQVPETTAQWLTTGFMLTMAVVIPTTGYLLDRFSTKTIFVTALLFFTVGTLTAALAPTFAVLLGARIIQAVGTALVMPLLMTVTLTVVPAERRGSMMGIISIVISVAPALGPTLSGVILNSLTWHWLFWMMLPIVAVALVIGFFLIKNIGETKITPLDVLSVILSVFAFGGLVYGFSSFGAILEGKGVVGIVAIVVGAIALVIFALRQHQLGKQDKALMDLRAFKVRNFSFSLTTILLAFGAMLGTVMVLPIYLQTSLGVTALVTGLVVMPGGLLQGLISPFIGRFYDKVGPRPLLIPGAIALAVAASSMTFLNENSPVWMVVVLHVIFSIGMCLMMTPLMTTALGALPKHLYGHGSAILNTFQQLAGAAGTAIMIAALSFGTSIAASSGSAHAEAVAAGTRVAFIAGAIIAVIALVVSLFVTRVEEETH

SEQ ID NO: 6 - protein, 481 aa, Corynebacterium glutamicum
  misc_feature (1)..(481): amino acid sequence disclosed in GenBank accession number KEI24239
  misc_feature (1)..(481): the encoded amino acid sequence of a protein from Corynebacterium glutamicum ATCC14067
MDSQINTQTSPAAAKLPREVVVVLSILVISAMIMILNETILSVALPSIMADFQVPETTAQWLTTGFMLTMAVVIPTTGYLLDRFSTKTIFVTALLFFTVGTLTAALAPTFAVLLGARIIQAVGTALVMPLLMTVTLTVVPAERRSSMMGIISIVISVAPALGPTLSGVILNSLTWHWLFWMMLPIVAVALVIGFFLIKNIGETKITPLDVLSVILSVFAFGGLVYGFSSFGAILEGEGTVGILAIVVGAIALLIFALRQHQLGKQDKALMDLRAFKVRNFSFSLTTILLAFGAMLGTVMVLPIYLQTSLGVTALVTGLVVMPGGLLQGLISPFIGRFYDKVGPRPLLIPGAIALAIAASSMTFLNENSPVWMVVVMHVVFSIGMCLMMTPLMTTALGALPKHLYGHGSAILNTFQQLAGAAGTAIMIAALSFGTSIAASSGSAHAEAVAAGTKVAFIAGAIIAVIALVISLFVTRVEEETH

SEQ ID NO: 7 - DNA, 3446 bp, Corynebacterium glutamicum
  misc_feature (1)..(3446): nucleotide sequence comprising locus_tag NCgl2592 disclosed in GenBank accession number NC_003450
  misc_feature (1)..(3446): the nucleotide sequence of the lmrB coding sequence of DM1933 including the nucleotide sequence upstream and downstream thereof, which is identical to that of ATCC13032
  misc_feature (1)..(1000): nucleotide sequence upstream of cds
  misc_feature (201)..(201): nucleobase adenine
  misc_feature (988)..(988): nucleobase cytosine
  CDS (1001)..(2443)
  misc_feature (1311)..(1311): nucleobase cytosine
  misc_feature (2444)..(2446): stop codon
  misc_feature (2444)..(3446): nucleotide sequence downstream of cds
  misc_feature (2456)..(2456): nucleobase adenine
  misc_feature (3246)..(3246): nucleobase thymine
caaatgattaaactcactcatgaagtcgcaatcaatgctgaaaagtaccgaagccttcctgcatgggataaagcagccgcccgagcttttgcagtgggtatgactgtggacaaagcagtggtagcggggcagtctgcggcgaggttatggggataccaaactttgactgttgagaaaaccgtgttgtgtctgttgccggaaaggctacgttcaaaatcctccaagcattggccgtccgggatgcgatataaagatcgttacctctcgtcgcgtgatattcgagaggttcatgggatccgagttacgggagcgttccgcacatttttggacatcgctttggatgatggggtggtggcggctgtggtcactattgattcagctcgaagacagaatccatcgcttacgcgtgagaagttaatgcacagtgcggaaagtttcccgaggcatcggggtgtgaaggcgtatcggcaggcgattgagttgtcgattcccaattcggatagtgctcaggagacgagggctcggttaatccttcgggaggccaagctcccggaaatccagtcagtgaaggtgcaggcccgtttcgatcaatcgcacaacaagtatttcctcgtcgatttcttgatcaatgagtggatcatcgtggagattgatggacgttcgaaatatgattccccggagctcaatgaggtgctcatggctgaacgcgatcgggagaaattcttcctcaatcagggctatgcggtcttaagaatcgatccgaaacagttagacctcaaccaagatggggagtgtgagttcatcggaatcctcaaaaacactttgcagaagaccccacctgagcacctcaagcaagccgcctaaacactccaagaaccaccatttgcacaatgcacttgcttgtggcactctttagtagttttttctcatagctcagtttcgcaactttagagaactctagaaactgagcttcatgctgtgaaaggccttttctccattcatggattcccaaattaatactcagacctctccggcagctgcgaagctgcctagggaggtcgttgttgttctttcgatcctcgtggtttccgcgatgatcatgattcttaatgaaaccattctgtcggttgcgttgccttccatcatggaagatttctccgtgcctgaaactactgcacagtggttgaccactggctttatgttgacgatggcagtggtgattccaactactggttatctgcttgatcgtttttccactaagacgatctttgttactgcgttgttgttctttacggttggtacgttgactgcggcgttggctccaacgtttgcggtgctgcttggtgctcgtatcgttcaggcggttggtactgcgctggtgatgcctttgctgatgacggttacgttgacggtggttcctgcggagcgtcgtggttcgatgatgggcattatttccatcgtgatttctgttgcgccggcgcttggtcctacgttgtctggtgtcattcttaactctttgacctggcactggttgttttggatgatgcttccgatcgttgttatcgctttggtaattggtttcttcttgatcaaaaatatcggcgaaaccaagatcaccccactggatgttctgtctgtcatcctttcggtgtttgccttcggtggtttggtgtacggcttcagttccttcggagcaatcctggagggcgaaggcaccgtaggtatcttcgcgatcgtcgttggcgccatcgcactcctcatctttgctttgcgacagcaccaactcggcaagcaagacaaagcactgatggatctccgagccttcaaggtgaggaacttcagcttctccttgaccaccatccttttggcgttcggcgcgatgctcggaaccgtcatggttttgccaatctacctgcagacttccctcggagttactgctttggtgaccggtttggttgttatgcccggcggcctcctccagggtctgatcagcccattcatcggacgtttctacgacaaggtcggtccacgtccgctgctgattcccggagcaattgcgctggctatcgcggcatcctcgatgactttcctcaatgagaattcacccgtgtggatggtcgtggtcatgcacgttgtgttcagcatcggcatgtgtttgatgatgacccctctcatgaccaccgctctcggcgcccttccgaagcacctctatggtcacggctccgcaattttgaacacgttccaacagctcgcaggcgcagccggaacagcgatcatgattgcagcactttccttcggcacttccattgcagcgtcttcgggatctgcgcatgctgaagctgttgccgctggtaccaaggttgcgttcatcgcaggcgcaatcatcgcggtgatcgctttggttgtttccctcttcgtcactcgcgtcgaggaagaagctcactaaataccaaaaaatggggcagaaggtcattgactttctgccccatttttttgcttctcgacgcctcccgccgccacctcaccttaagcgtgctcagcgctccaggttctgttctcgccacccggtttgtggttcccacaccaacagcttgttggtaggtcggctttctgggtttttcagtgaaataacaattttggttcgggaagaatatgccactgaaaaactattcctgtctaagttcacagttgttatttgttctgttgtttcagggtcgtggaaagcatatgtgccttcttgctcatgaaacatcatgagtgttccggtgggaaaaccgagtgttggagaatccccaggtggaagataaattagaccagattcgggaacctcagggtcgccaaagacttctacgttgagaatctcgaggtcctggttaaacgtgatgtggtggttactagcgcgtaagtaaattcgatcaccgtatgggtaaattgctgtgattatcgatcggccgatatcaagatctactgctttaaatggtttaagcctaaggattgtttgaagccaaaagttgtcattaagacgagggttttcgcgtgtgacattgtgcaagtttttcccgaagctgtctaacaatctccaacctgctggaatcgaagggacagcttcgaatacttctttaagatccggaagctcccatgtttttcccttgcaatcactgaaatcgtgaggccattgcttttgttttacgcgggctctattagcggaatcgaggacgatctcttggaacgtggggatggagatctcgtgtaatagttcgcctgttgacacgctgaatccacggacgaaaggcaagctttcatcggagagccaaaatatgtcaccgtcactggtactagtgctgatgccttcaagagtttcttggtttttgggtggatgcacaccatctagggtcacgtcgataattgcgtccgtaccgtggtagtgcgctgctgtaacgactgca

SEQ ID NO: 8 - protein, 481 aa, Corynebacterium glutamicum; identical to SEQ ID NO: 1
  misc_feature (1)..(481): a strain of Corynebacterium glutamicum, identical to SEQ ID NO2 of WO2001000804A2

SEQ ID NO: 9 - DNA, 3446 bp, Corynebacterium glutamicum
  misc_feature (1)..(3446): nucleotide sequence comprising locus_tag BBD29_13115 disclosed in GenBank accession number NZ_CP016335
  misc_feature (1001)..(2443): a nucleotide sequence encoding the LmrB polypeptide from ATCC13869
  misc_feature (1)..(1000): nucleotide sequence upstream of cds
  CDS (1001)..(2443)
  misc_feature (2444)..(2446): stop codon
  misc_feature (2444)..(3446): nucleotide sequence downstream of cds
caaatgattaaactcactcatgaagtcgcaatcaatgctgaaaagtaccgaagccttcctgcatgggataaagcagccgcccgagcttttgcagtgggtatgactgtggacaaagcagtggtagcggggcagtctgcggcgaggttatggggataccaaactttgactgttgagaaaaccgtgttgtgtctgttgccggaaaggctacgttcaaaatcctccaagcattggccgtccgggatgcgatataaagatcgttacctctcgccgcgtgatattcgggaagttcatgggattcgagtcacgggagctttccgcacatttttggacatcgctttggatgatggggtggtggcggctgtggtcactattgattcagctcgaaggcagaatccgtcgcttacgcgtgagaagttgatgtgcagtgcagaaagtttcccgaggcatcggggtgtgaaggcgtatcggcaggcgattgagttgtcgattcccaattcggatagtgctcaggagacgagggctcggttgatcctgggggaggccaacctcccggagatccagtcagtgcaggtgcaagcccgtttcgatcaatcgcacaacaagtatttcctcgtggatttcttaatcaatgagtggatcatcgtggagattgatggacgttcgaaatatgattctccagagcttaatgaggtgctcatggctgaacgtgatcgggagaaattcttcctcaaccaaggatatgcagtgctgagaattgatccaaaacaattagaccccgaccaaaacggcgagtgtgaattcatcggaatcctcaaaaacactttgcagaagaccccgccagagcacctcaagcaagccgcctaaacactccaagaaccaccatttgcacaatgcacttgcttgtggcactctttagtagttttttctcatagctcagtttcgcaactttagagaactctagaaactgagcttcatgctgtgaaaggccttttctccattcatggattcccaaattaatactcagacctctccggcagctgcgaagctgcctagggaggtcgttgttgttctttcgatcctcgtggtttccgcgatgatcatgattcttaatgaaaccattctgtcggttgcgttgccttccatcatggaagatttctccgtgcctgaaactactgcacagtggttgaccactggctttatgttgacgatggcagtggtgattcctactactggttatctgcttgatcgtttttccactaagacgatctttgttactgcgttgttgttctttacggttggtacgttgactgcggcgttggccccaacgtttgctgtgctgcttggtgctcgtatcgttcaggcggttggtactgcgctggtgatgcctttgctgatgacagttacgttgacggtggttcctgcggagcgtcgtggttcgatgatgggcattatttccatcgtgatttctgttgcgccggcgcttggtcctactttgtctggtgtcattcttaactctttgacctggcactggttgttttggatgatgcttccgatcgttgttatcgctttggtgattggctacttcctgatccaaaacattggcgaaaccaagatcactccactggatgttctgtctgtcatcctttcggtgtttgccttcggtggtttggtgtacggcttcagttccttcggagcaatcctggagggcgaaggtaccgtaggtatcctcgcgatcgtcgttggcgccatcgcacttctcatctttgctttgcgacagcaccaacttggcaaacaagacaaagccctcatggatttgcgtgctttcaaggtgaggaacttcagtttctccttgaccaccatccttttggcgttcggtgcgatgctcggaaccgtcatggttttgcccatctacctgcagacttccctcggagttactgctttggtgaccggtttggttgttatgcccggcggcctcctccagggtctgatcagcccattcatcggacgtttctacgacaaggtcggtccacgtccgctgctgattcccggagcaattgcgctggctatcgcggcatcctcgatgactttcctcaatgagaattcacctgtgtggatggtcgtggtcatgcacgttgtgttcagcatcggtatgtgtttgatgatgacccctctcatgaccaccgctctcggcgcccttccgaagcacctctatggtcacggctccgcaattttgaacacgttccaacagctcgcaggtgcagccggaacagcgatcatgattgcagcactttccttcggcacttccattgcagcgtcttccggatctgcgcatgctgaagctgttgccgctggtaccaaggttgcgttcatcgcaggcgcaatcatcgcggtgatcgctttggttgtttccctcttcgtcactcgcgttgaggaagaaactcactaaacgctcaataatggggcagacggtcattgactttctgccccggttttttgcttttcgacgcctccctgcagcgcaccttgcaggtgcgcggggcttaagcgtcgaaagccctagatttctacagagacatcgtcgaatttaatgttgctccattgaccgtctctaaacactgagacagaggaaggtactggtttgagataatcagtaccagtaggcggggtaaccagtatgttgaagtggtttggtgacagaattctgacctcggtgaagtacccttccggtgttgttcacctggttatttcgcttcctgaaatctgatcgagaaatacgatttcatcttttttcgtgaacttggcagctacgccttcttgtgtaatccagcgttcatgcttcgcaccaagctgatgttcttcggcactttgcacttgaaagtttgaatccaaaaagacggtcatgctccagaattgcacaaagtacaggtcttctactcggcatgcgtttaggatcctggatccgtcattgctgattacacagatttccaattgtcctgctggagtgcttcgacccagtgctgtttgctctccatcggtcagggagtaaagcccatcgagaaagctttcttgtaattcccaaggcgctggaacgtcgagttctatgacaggctctatccatggttgaggttccgcaagcatgcgtactctaggattcagtgaccaggcgttttcataatcgtgaatcaacccttccgaaaactgaagcggagacccgtgagaaaccgaaataggcacataggtttgccccaggtattttccggtttggagatcccagcatcgaagaattggaagcatctgatcaaggacccatagttgttcattgatgatgtgagtgatggatgcttcaaactgttgtggctgaagtggtggtggaactgtgccgtcaagggtggcatcgacgataacttccaacagccagctgccattttctgtgtaggcatcgtctg

SEQ ID NO: 10 - protein, 481 aa, Corynebacterium glutamicum; identical to SEQ ID NO: 3
  misc_feature (1)..(481): an amino acid sequence of the encoded LmrB polypeptide from ATCC13869

SEQ ID NO: 11 - DNA, 3446 bp, Corynebacterium glutamicum
  misc_feature (1)..(3446): nucleotide sequence comprising locus_tag KIQ_001365 disclosed in GenBank accession number AGQQ02000002
  misc_feature (1001)..(2443): a nucleotide sequence encoding the LmrB polypeptide from ATCC14067
  misc_feature (1)..(1000): nucleotide sequence upstream of cds
  CDS (1001)..(2443)
  misc_feature (2444)..(2446): stop codon
  misc_feature (2444)..(3446): nucleotide sequence downstream of cds
tacccttccctcaacgaagccctcgcacgcaaattcccaggacaaccagaagtaaccatcgactccaccgtcgaagaactccgcgaaatcgccaaggtaatcggcctctccgtccccgaaaatggcggctggggacacggcaaactcgtcgaagaaatctgggaactcctctgcgaagaccaactctacggaccaatctttgtcaaagacttcccagtagaaacctccccactcacacgccaacaccgcaccaagccaggcgtcaccgaaaagtgggacctctacgtccgcggatttgaactagcaaccggatactccgaactcatcgacccagtcatccaacgcgaacgcttcgaagaccaagcccgcctcgccgccgacggagacgacgaagccatggtcctcgacgaagacttcctcaccgcaatggaacaaggcatgccaccaacctccggcaacggcatgggaatcgaccgcctcctcatggccctcaccggcctcggaatccgcgaaaccgtactcttcccaatggtgaaaccagaacaaaagtaggttttctgtttgggtaggttagctccaggactttgtgtcctggagttttcctcttcctagggttgatcgggatgggttttgtaaggtcgggtcggcgtcggtccgtaagttcgatttcacggtttcgagaccttaacagcgatttcacgggtttcggtttctcgcgttcaatttcttgtactcaatttcgcatattcgataccactaggaaagacgtcaaaatccgttagacctcgaccaaaacggcgagtgcgagttcatcggaatcctcaaaaacactttgcagaagaccccaccagagcacctcaggcaagccgcctaaacactccaagaaccaccacttgcacaatgcacttgcttatggcactctttaggagttttttctcatagctcagtttcgtaacactagaaactgagcttcatgctgtgaaaggctttttctccattcatggattcccaaattaatactcagacctctccggcagctgcgaagttgcctagggaggtcgttgttgttctttcgatcctcgtgatttctgcgatgatcatgattcttaatgaaactatcctgtcggtcgcgttgccgtcgatcatggcggatttccaggttccagaaactactgcgcagtggttgaccactggctttatgttgacgatggcagtggtgattcctactactggttatttgcttgatcgtttttccactaagacgatctttgttactgcgttgttgttctttactgttggtaccttgactgcggcgttggccccaacgtttgctgtgctgcttggtgctcgtatcattcaggcggttggtactgcgctggtgatgcctttgctaatgacagtcacgttgacggtggttcctgcggagcggcgtagttcgatgatgggcattatttccatcgtgatttctgtcgcgccggcgcttggtcctactttgtctggtgtcattcttaactctttgacctggcactggttgttctggatgatgctcccgatcgtggccgttgcgctggttattggcttctttttgatcaaaaatatcggcgaaaccaagatcaccccactggatgttctgtctgtcatcctttcggtgtttgccttcggtggtttggtgtacggattcagttccttcggagcaatcctggagggcgaaggcaccgtaggtatcctcgcgatcgtcgttggcgccatcgcacttctcatctttgctttgcgacagcaccaactcggcaaacaagacaaggcactgatggatctccgtgctttcaaggtgaggaacttcagcttctccttgaccaccatccttttggcgttcggcgcgatgctcggaaccgtcatggttttgccaatctacctgcagacttctctcggagttactgctttggtgaccggtttggttgttatgcccggcggcctcctccagggtctgatcagcccattcatcggacgtttctacgacaaggttggtccacgtccgttgctgattcccggagcaattgcgctggctatcgcggcatcctcgatgactttcctcaatgagaattcacctgtgtggatggtcgtggtcatgcacgttgtgttcagcatcggcatgtgtttgatgatgacccctctcatgaccaccgctctcggcgcccttccgaagcacctctatggtcacggctccgcaattctgaacacgttccaacagctcgcaggcgcagccggaacagcgatcatgattgcggcactttccttcggcacttccattgcagcgtcttccggatctgcacacgccgaagctgtcgccgctggcaccaaggttgcgttcatcgcaggcgcaatcatcgcagtgatcgctttggttatttccctcttcgtcactcgcgtcgaggaagaaactcactaaacaccaaaaaatggggcagaaggtcattgactttctgccccattttttgcttctcgacgcctcccgccgccacctcaccttaagcgtgctcagcgctccaggttttgttctcgccacccggtttgtggttcccacaccaacagtttgtcaatgaggcggctttctggatttttcagcgaaataacaattctgttctgagaagcatattccacttcaaactgccttttgggcaagttcacagttgttagttgctttgttgttttaggatcgtggaaagcgtagatatcttgttgttcgtggaacatcatgagtgatccgataggaaatcccagcgttggagaatcccctggtggaagatcggaaagtggccaataacctgcatcgggatttccgtggacctctacactgaggatttcaagatcctgattaaacgtgatctggtgaagatcggaacgcagataaattcgttcaccgtattggtaaatcgtcgaaatctcggcgtatccaagatcaagctcaattgctttgaagggtttgatcctaaggatggtttgtttccaggttccgttgtcagcgcttacgacatggaagttcttcccgaatcttttgtgtagtttccagcctgcggggatcgctgggacagggtcggttgcctctttaaggtctggaagttcccaaagttttttcgctgcgcgggctctattgccggattcgagggcgatctcattgaaggtgggtatggagatctcgtgtactagcttgcctgtggagacattgaaaccgcggacgaagggcaagctcttatcggacagccagaatgtttcaccgtcacaggtactggtgctgctccaatccaaggattcttgatatctaggaggtttcgcaccgtcgagggtcacgtcgataattgcgtccttaccgtggcggtagactgcggtgatgaccgctgacgtgcggcggctcgtgtccgcgtaggagctgtgggtgaagtagccggtga

SEQ ID NO: 12 - protein, 481 aa, Corynebacterium glutamicum; identical to SEQ ID NO: 6
  misc_feature (1)..(481): a polynucleotide called lmrBATCC14067 encoding the LmrB polypeptide of strain ATCC14067

SEQ ID NO: 13 - DNA, 1585 bp, artificial sequence
  nucleotide sequence comprising a deletion of the cds of the lmrB gene of Corynebacterium glutamicum ATCC13032 and the adjoining stop codon accompanied by insertion of the recognition site for restriction endonuclease EcoRV
  misc_feature (1)..(788): nucleotide sequence of ATCC13032 upstream (5′-flanking sequence) of sequence coding for LmrB polypeptide
  misc_feature (789)..(794): artificially inserted recognition site for restriction endonuclease EcoRV
  misc_feature (795)..(1585): nucleotide sequence of ATCC13032 downstream (3′-flanking sequence) of sequence coding for LmrB polypeptide
aaggctacgttcaaaatcctccaagcattggccgtccgggatgcgatataaagatcgttacctctcgtcgcgtgatattcgagaggttcatgggatccgagttacgggagcgttccgcacatttttggacatcgctttggatgatggggtggtggcggctgtggtcactattgattcagctcgaagacagaatccatcgcttacgcgtgagaagttaatgcacagtgcggaaagtttcccgaggcatcggggtgtgaaggcgtatcggcaggcgattgagttgtcgattcccaattcggatagtgctcaggagacgagggctcggttaatccttcgggaggccaagctcccggaaatccagtcagtgaaggtgcaggcccgtttcgatcaatcgcacaacaagtatttcctcgtcgatttcttgatcaatgagtggatcatcgtggagattgatggacgttcgaaatatgattccccggagctcaatgaggtgctcatggctgaacgcgatcgggagaaattcttcctcaatcagggctatgcggtcttaagaatcgatccgaaacagttagacctcaaccaagatggggagtgtgagttcatcggaatcctcaaaaacactttgcagaagaccccacctgagcacctcaagcaagccgcctaaacactccaagaaccaccatttgcacaatgcacttgcttgtggcactctttagtagttttttctcatagctcagtttcgcaactttagagaactctagaaactgagcttcatgctgtgaaaggccgatatcaatggggcagaaggtcattgactttctgccccatttttttgcttctcgacgcctcccgccgccacctcaccttaagcgtgctcagcgctccaggttctgttctcgccacccggtttgtggttcccacaccaacagcttgttggtaggtcggctttctgggtttttcagtgaaataacaattttggttcgggaagaatatgccactgaaaaactattcctgtctaagttcacagttgttatttgttctgttgtttcagggtcgtggaaagcatatgtgccttcttgctcatgaaacatcatgagtgttccggtgggaaaaccgagtgttggagaatccccaggtggaagataaattagaccagattcgggaacctcagggtcgccaaagacttctacgttgagaatctcgaggtcctggttaaacgtgatgtggtggttactagcgcgtaagtaaattcgatcaccgtatgggtaaattgctgtgattatcgatcggccgatatcaagatctactgctttaaatggtttaagcctaaggattgtttgaagccaaaagttgtcattaagacgagggttttcgcgtgtgacattgtgcaagtttttcccgaagctgtctaacaatctccaacctgctggaatcgaagggacagcttcgaatacttctttaagatccggaagctcccatgtttttcccttgcaatcactgaaatcgtgaggccattgcttttgttttacgcgggctctattagcggaatcgaggacgatctcttggaacgtggggatggagatct

SEQ ID NO: 14 - DNA, 1585 bp, artificial sequence
  nucleotide sequence comprising a deletion of the cds of the lmrB gene of Corynebacterium glutamicum ATCC14067 and the adjoining stop codon accompanied by insertion of the recognition site for restriction endonuclease EcoRV
  misc_feature (1)..(788): nucleotide sequence of ATCC14067 upstream (5′-flanking sequence) of sequence coding for LmrB polypeptide
  misc_feature (789)..(794): artificially inserted recognition site for restriction endonuclease EcoRV
  misc_feature (789)..(794): nucleotide sequence of ATCC14067 downstream (3′-flanking sequence) of sequence coding for LmrB polypeptide
tgtcaaagacttcccagtagaaacctccccactcacacgccaacaccgcaccaagccaggcgtcaccgaaaagtgggacctctacgtccgcggatttgaactagcaaccggatactccgaactcatcgacccagtcatccaacgcgaacgcttcgaagaccaagcccgcctcgccgccgacggagacgacgaagccatggtcctcgacgaagacttcctcaccgcaatggaacaaggcatgccaccaacctccggcaacggcatgggaatcgaccgcctcctcatggccctcaccggcctcggaatccgcgaaaccgtactcttcccaatggtgaaaccagaacaaaagtaggttttctgtttgggtaggttagctccaggactttgtgtcctggagttttcctcttcctagggttgatcgggatgggttttgtaaggtcgggtcggcgtcggtccgtaagttcgatttcacggtttcgagaccttaacagcgatttcacgggtttcggtttctcgcgttcaatttcttgtactcaatttcgcatattcgataccactaggaaagacgtcaaaatccgttagacctcgaccaaaacggcgagtgcgagttcatcggaatcctcaaaaacactttgcagaagaccccaccagagcacctcaggcaagccgcctaaacactccaagaaccaccacttgcacaatgcacttgcttatggcactctttaggagttttttctcatagctcagtttcgtaacactagaaactgagcttcatgctgtgaaaggctgatatcaatggggcagaaggtcattgactttctgccccattttttgcttctcgacgcctcccgccgccacctcaccttaagcgtgctcagcgctccaggttttgttctcgccacccggtttgtggttcccacaccaacagtttgtcaatgaggcggctttctggatttttcagcgaaataacaattctgttctgagaagcatattccacttcaaactgccttttgggcaagttcacagttgttagttgctttgttgttttaggatcgtggaaagcgtagatatcttgttgttcgtggaacatcatgagtgatccgataggaaatcccagcgttggagaatcccctggtggaagatcggaaagtggccaataacctgcatcgggatttccgtggacctctacactgaggatttcaagatcctgattaaacgtgatctggtgaagatcggaacgcagataaattcgttcaccgtattggtaaatcgtcgaaatctcggcgtatccaagatcaagctcaattgctttgaagggtttgatcctaaggatggtttgtttccaggttccgttgtcagcgcttacgacatggaagttcttcccgaatcttttgtgtagtttccagcctgcggggatcgctgggacagggtcggttgcctctttaaggtctggaagttcccaaagttttttcgctgcgcgggctctattgccggattcgagggcgatctcattgaaggtgggtatggagatctcgtgtactagcttgcctgtggagacattgaaaccgcggacgaagggcaagct

SEQ ID NO: 15 - DNA, 1266 bp, Corynebacterium glutamicum
  misc_feature (1)..(1266): a nucleotide sequence of the coding sequence of the aspartokinase polypeptide of strain ATCC13032
  CDS (1)..(1263): nucleotide sequence encoding aspartokinase
  misc_feature (1)..(3): gtg start codon
  misc_feature (1264)..(1266): stop codon
gtggccctggtcgtacagaaatatggcggttcctcgcttgagagtgcggaacgcattagaaacgtcgctgaacggatcgttgccaccaagaaggctggaaatgatgtcgtggttgtctgctccgcaatgggagacaccacggatgaacttctagaacttgcagcggcagtgaatcccgttccgccagctcgtgaaatggatatgctcctgactgctggtgagcgtatttctaacgctctcgtcgccatggctattgagtcccttggcgcagaagcccaatctttcacgggctctcaggctggtgtgctcaccaccgagcgccacggaaacgcacgcattgttgatgtcactccaggtcgtgtgcgtgaagcactcgatgagggcaagatctgcattgttgctggtttccagggtgttaataaagaaacccgcgatgtcaccacgttgggtcgtggtggttctgacaccactgcagttgcgttggcagctgctttgaacgctgatgtgtgtgagatttactcggacgttgacggtgtgtataccgctgacccgcgcatcgttcctaatgcacagaagctggaaaagctcagcttcgaagaaatgctggaacttgctgctgttggctccaagattttggtgctgcgcagtgttgaatacgctcgtgcattcaatgtgccacttcgcgtacgctcgtcttatagtaatgatcccggcactttgattgccggctctatggaggatattcctgtggaagaagcagtccttaccggtgtcgcaaccgacaagtccgaagccaaagtaaccgttctgggtatttccgataagccaggcgaggctgcgaaggttttccgtgcgttggctgatgcagaaatcaacattgacatggttctgcagaacgtctcttctgtagaagacggcaccaccgacatcaccttcacctgccctcgttccgacggccgccgcgcgatggagatcttgaagaagcttcaggttcagggcaactggaccaatgtgctttacgacgaccaggtcggcaaagtctccctcgtgggtgctggcatgaagtctcacccaggtgttaccgcagagttcatggaagctctgcgcgatgtcaacgtgaacatcgaattgatttccacctctgagattcgtatttccgtgctgatccgtgaagatgatctggatgctgctgcacgtgcattgcatgagcagttccagctgggcggcgaagacgaagccgtcgtttatgcaggcaccggacgctaa

SEQ ID NO: 16 - protein, 421 aa, Corynebacterium glutamicum
  misc_feature (1)..(421): the amino acid sequence of the encoded polypeptide from strain ATCC13032
MALVVQKYGGSSLESAERIRNVAERIVATKKAGNDVVVVCSAMGDTTDELLELAAAVNPVPPAREMDMLLTAGERISNALVAMAIESLGAEAQSFTGSQAGVLTTERHGNARIVDVTPGRVREALDEGKICIVAGFQGVNKETRDVTTLGRGGSDTTAVALAAALNADVCEIYSDVDGVYTADPRIVPNAQKLEKLSFEEMLELAAVGSKILVLRSVEYARAFNVPLRVRSSYSNDPGTLIAGSMEDIPVEEAVLTGVATDKSEAKVTVLGISDKPGEAAKVFRALADAEINIDMVLQNVSSVEDGTTDITFTCPRSDGRRAMEILKKLQVQGNWTNVLYDDQVGKVSLVGAGMKSHPGVTAEFMEALRDVNVNIELISTSEIRISVLIREDDLDAAARALHEQFQLGGEDEAVVYAGTGR

SEQ ID NO: 17 - DNA, 41 bp, artificial sequence
  primer
  misc_feature (1)..(41): primer 1f_DlmrB
agctcggtacccggggatcctagatctccatccccacgttc

SEQ ID NO: 18 - DNA, 46 bp, artificial sequence
  primer
  misc_feature (1)..(46): primer 1r_DlmrB
gcttcatgctgtgaaaggccgatatcaatggggcagaaggtcattg

SEQ ID NO: 19 - DNA, 46 bp, artificial sequence
  primer
  misc_feature (1)..(46): primer 2f_DlmrB
caatgaccttctgccccattgatatcggcctttcacagcatgaagc

SEQ ID NO: 20 - DNA, 39 bp, artificial sequence
  primer
  misc_feature (1)..(39): primer 2r_DlmrB
tgcatgcctgcaggtcgactaaggctacgttcaaaatcc

SEQ ID NO: 21 - DNA, 834 bp, artificial sequence
  amplificate lmrB_up
  misc_feature (1)..(20): 5′-overlap to pK18mobsacB cut by XbaI
  misc_feature (21)..(808): sequence corresponding to the sequence of SEQ ID NO7 positions 201 to 988
  misc_feature (789)..(834): 3′-overlap to amplificate lmrB_down
  misc_feature (809)..(814): recognition site for restriction endonuclease EcoRV
tgcatgcctgcaggtcgactaaggctacgttcaaaatcctccaagcattggccgtccgggatgcgatataaagatcgttacctctcgtcgcgtgatattcgagaggttcatgggatccgagttacgggagcgttccgcacatttttggacatcgctttggatgatggggtggtggcggctgtggtcactattgattcagctcgaagacagaatccatcgcttacgcgtgagaagttaatgcacagtgcggaaagtttcccgaggcatcggggtgtgaaggcgtatcggcaggcgattgagttgtcgattcccaattcggatagtgctcaggagacgagggctcggttaatccttcgggaggccaagctcccggaaatccagtcagtgaaggtgcaggcccgtttcgatcaatcgcacaacaagtatttcctcgtcgatttcttgatcaatgagtggatcatcgtggagattgatggacgttcgaaatatgattccccggagctcaatgaggtgctcatggctgaacgcgatcgggagaaattcttcctcaatcagggctatgcggtcttaagaatcgatccgaaacagttagacctcaaccaagatggggagtgtgagttcatcggaatcctcaaaaacactttgcagaagaccccacctgagcacctcaagcaagccgcctaaacactccaagaaccaccatttgcacaatgcacttgcttgtggcactctttagtagttttttctcatagctcagtttcgcaactttagagaactctagaaactgagcttcatgctgtgaaaggccgatatcaatggggcagaaggtcattg

SEQ ID NO: 22 - DNA, 838 bp, artificial sequence
  amplificate lmrB_down
  misc_feature (1)..(46): 5′-overlap to amplificate lmrB_up
  misc_feature (21)..(26): recognition site for restriction endonuclease EcoRV
  misc_feature (27)..(817): sequence corresponding to the sequence of SEQ ID NO7 positions 2456 to 3246
  misc_feature (818)..(838): 3′-overlap to pK18mobsacB cut by XbaI
gcttcatgctgtgaaaggccgatatcaatggggcagaaggtcattgactttctgccccatttttttgcttctcgacgcctcccgccgccacctcaccttaagcgtgctcagcgctccaggttctgttctcgccacccggtttgtggttcccacaccaacagcttgttggtaggtcggctttctgggtttttcagtgaaataacaattttggttcgggaagaatatgccactgaaaaactattcctgtctaagttcacagttgttatttgttctgttgtttcagggtcgtggaaagcatatgtgccttcttgctcatgaaacatcatgagtgttccggtgggaaaaccgagtgttggagaatccccaggtggaagataaattagaccagattcgggaacctcagggtcgccaaagacttctacgttgagaatctcgaggtcctggttaaacgtgatgtggtggttactagcgcgtaagtaaattcgatcaccgtatgggtaaattgctgtgattatcgatcggccgatatcaagatctactgctttaaatggtttaagcctaaggattgtttgaagccaaaagttgtcattaagacgagggttttcgcgtgtgacattgtgcaagtttttcccgaagctgtctaacaatctccaacctgctggaatcgaagggacagcttcgaatacttctttaagatccggaagctcccatgtttttcccttgcaatcactgaaatcgtgaggccattgcttttgttttacgcgggctctattagcggaatcgaggacgatctcttggaacgtggggatggagatctaggatccccgggtaccgagct

SEQ ID NO: 23 - DNA, 20 bp, artificial sequence
  primer
  misc_feature (1)..(20): primer pCV22_1.p
aggtttcccgactggaaagc

SEQ ID NO: 24 - DNA, 20 bp, artificial sequence
  primer
  misc_feature (1)..(20): primer pCV22_2.p
tgcaaggcgattaagttggg

SEQ ID NO: 25 - DNA, 23 bp, artificial sequence
  primer
  misc_feature (1)..(23): primer pVW_1.p
gtgagcggataacaatttcacac

SEQ ID NO: 26 - DNA, 16 bp, artificial sequence
  primer
  misc_feature (1)..(13): primer M13For
gtaaaacgacggccag

SEQ ID NO: 27 - DNA, 20 bp, artificial sequence
  primer
  misc_feature (1)..(20): primer NCgl2591_fw
ggcgagaacagaacctggag

SEQ ID NO: 28 - DNA, 20 bp, artificial sequence
  primer
  misc_feature (1)..(20): primer NCgl2591_rev
ctcaagcaagccgcctaaac

SEQ ID NO: 29 - DNA, 20 bp, artificial sequence
  primer
  misc_feature (1)..(20): primer NCgl2591_fwd1
caagagatcgtcctcgattc

SEQ ID NO: 30 - DNA, 20 bp, artificial sequence
  primer
  misc_feature (1)..(20): primer NCgl2593_rev1
gtccgggatgcgatataaag

SEQ ID NO: 31 - DNA, 20 bp, artificial sequence
  primer
  misc_feature (1)..(20): primer NCgl2591_fwd2
ttccagcaggttggagattg

SEQ ID NO: 32 - DNA, 20 bp, artificial sequence
  primer
  misc_feature (1)..(20): primer NCgl2593_rev2
ggcggctgtggtcactattg

SEQ ID NO: 33 - DNA, 1532 bp, Corynebacterium glutamicum
  misc_feature (1)..(1532): sequence displayed in SEQ ID NO13 positions 34 to 1565
  misc_feature (1)..(1532): a strain of Corynebacterium glutamicum known as DM1933, where the ImrB gene of strain DM1933 is replaced with the deltalmrB::EcoRV mutation
  misc_feature (756)..(761): recognition site for restriction endonuclease EcoRV
gtccgggatgcgatataaagatcgttacctctcgtcgcgtgatattcgagaggttcatgggatccgagttacgggagcgttccgcacatttttggacatcgctttggatgatggggtggtggcggctgtggtcactattgattcagctcgaagacagaatccatcgcttacgcgtgagaagttaatgcacagtgcggaaagtttcccgaggcatcggggtgtgaaggcgtatcggcaggcgattgagttgtcgattcccaattcggatagtgctcaggagacgagggctcggttaatccttcgggaggccaagctcccggaaatccagtcagtgaaggtgcaggcccgtttcgatcaatcgcacaacaagtatttcctcgtcgatttcttgatcaatgagtggatcatcgtggagattgatggacgttcgaaatatgattccccggagctcaatgaggtgctcatggctgaacgcgatcgggagaaattcttcctcaatcagggctatgcggtcttaagaatcgatccgaaacagttagacctcaaccaagatggggagtgtgagttcatcggaatcctcaaaaacactttgcagaagaccccacctgagcacctcaagcaagccgcctaaacactccaagaaccaccatttgcacaatgcacttgcttgtggcactctttagtagttttttctcatagctcagtttcgcaactttagagaactctagaaactgagcttcatgctgtgaaaggccgatatcaatggggcagaaggtcattgactttctgccccatttttttgcttctcgacgcctcccgccgccacctcaccttaagcgtgctcagcgctccaggttctgttctcgccacccggtttgtggttcccacaccaacagcttgttggtaggtcggctttctgggtttttcagtgaaataacaattttggttcgggaagaatatgccactgaaaaactattcctgtctaagttcacagttgttatttgttctgttgtttcagggtcgtggaaagcatatgtgccttcttgctcatgaaacatcatgagtgttccggtgggaaaaccgagtgttggagaatccccaggtggaagataaattagaccagattcgggaacctcagggtcgccaaagacttctacgttgagaatctcgaggtcctggttaaacgtgatgtggtggttactagcgcgtaagtaaattcgatcaccgtatgggtaaattgctgtgattatcgatcggccgatatcaagatctactgctttaaatggtttaagcctaaggattgtttgaagccaaaagttgtcattaagacgagggttttcgcgtgtgacattgtgcaagtttttcccgaagctgtctaacaatctccaacctgctggaatcgaagggacagcttcgaatacttctttaagatccggaagctcccatgtttttcccttgcaatcactgaaatcgtgaggccattgcttttgttttacgcgggctctattagcggaatcgaggacgatctcttg

SEQ ID NO: 34 - DNA, 37 bp, artificial sequence
  primer
  misc_feature (1)..(37): primer 1f-lmrB
gcctgcaggtcgactatggattcccaaattaatactc

SEQ ID NO: 35 - DNA, 35 bp, artificial Sequence
  primer
  misc_feature (1)..(35): primer 1r-lmrB
ggtacccggggatcctttagtgagtttcttcctcg

SEQ ID NO: 36 - DNA, 20 bp, artificial sequence
  primer
  misc_feature (1)..(20): primer pVW_2.p
cgacggccagtgaattcgag

SEQ ID NO: 37 - DNA, 35 bp, artificial sequence
  primer
  misc_feature (1)..(35): primer pVT_rev
ggtacccggggatcctttagtgagcttcttcctcg